(19) United States
(12) Patent Application Publication
(10) Pub. No.: US 2025/0346890 A1
(43) Pub. Date: Nov. 13, 2025

(54) LEUKEMIA TREATMENT

(71) Applicant: Deutsches Krebsforschungszentrum, Heidelberg (DE)

(72) Inventors: Christof Niehrs, Heidelberg (DE); Rui Sun, Heidelberg (DE); Hyeyoon Lee, Heidelberg (DE)

(21) Appl. No.: 17/787,855

(22) PCT Filed: Dec. 18, 2020
(86) PCT No.: PCT/EP2020/087065

§ 371 Date: Jun. 21, 2022

(30) Foreign Application Priority Data
Dec. 19, 2019 (EP) 19218160.0

(51) Int. Cl. C12N 15/113; A61P 35/02; C07K 16/18; G01N 33/50; G01N 33/574

(52) U.S. Cl. CPC C12N 15/113; A61P 35/02; C07K 16/18; G01N 33/5011; G01N 33/57426; C07K 2317/76; C12N 2310/14; C12N 2310/531

(57) ABSTRACT

The present invention relates to an inhibitor of R-spondin 2 and/or R-spondin 3 mediated bone morphogenetic protein (BMP) receptor inhibition for use in treating and/or preventing leukemia in a subject; and to methods, kits, combined preparations, and uses related thereto.

### CROSS-REFERENCE TO RELATED APPLICATIONS

This application is a U.S. National Phase of International PCT Application No. PCT/EP2020/087065 filed on Dec. 18, 2020, which claims priority to European Patent Application No. 19218160.0 filed on Dec. 19, 2019, the contents of each application are incorporated herein by reference in their entireties.

The present invention relates to an inhibitor of R-spondin 2 and/or R-spondin 3 mediated bone morphogenetic protein (BMP) receptor inhibition for use in treating and/or preventing leukemia in a subject; and to methods, kits, combined preparations, and uses related thereto.

R-Spondins (“roof plate-specific spondins”, RSPO1-4) are a family of four secreted ˜30 kDa proteins implicated in development and cancer (Hao et al. (2016), Cancers (Basel) 8, doi: 10.3390/cancers8060054). RSPOs are a key ingredient to maintain organoid cultures where they stimulate stem cell growth (Sato et al. (2009), Nature 459, 262-265, doi: 10.1038/nature07935). They amplify WNT signaling by preventing Frizzled/LRP5/6 receptor ubiquitination and degradation via transmembrane E3 ubiquitin ligases ring finger 43 (RNF43) and zinc and ring finger 3 (ZNRF3), thereby sensitizing cells to WNT ligands. RSPOs bind to ZNRF3/RNF43 and to the stem cell marker Leucine-rich repeat containing G protein-coupled receptor 5 (LGR5), and two related proteins, LGR4 and LGR6, leading to the internalization of the RSPO-LGR-ZNRF3/RNF43 complex and lysosomal degradation. RSPOs harbor a signal peptide, two adjacent cysteine-rich furin-like (FU) domains, a thrombospondin I repeat (TSR) domain, and a basic amino acid-rich (BR) domain with varying length at the C-terminus; the two furin-like repeats (FU1, FU2) domains bind to ZNRF3/RNF43 and LGRs, respectively. The TSP1 domain possess about 40% overall sequence homology between RSPOs. The TSP1 domain is not essential for WNT/LRP6 signaling but it binds to HSPGs (Heparan Sulfate Proteoglycans) and thereby promotes WNT5A/PCP (planar cell polarity) signaling.

Bone morphogenetic protein (BMP) receptors are a family of transmembrane serine/threonine kinases closely related to activin receptors. The ligands of BMP receptors are members of the TGF beta superfamily.

Acute myeloid leukemia (AML) is a type of leukemia arising from uncontrolled proliferation and impaired differentiation of myeloid precursors (Nowak et al. (2009), Blood 113, 3655-3665, doi: 10.1182/blood-2009-01-198911).

In accordance, the present invention relates to an inhibitor of R-spondin 2 and/or R-spondin 3 mediated bone morphogenetic protein (BMP) receptor inhibition for use in treating and/or preventing leukemia in a subject. In an embodiment, the present invention relates to an inhibitor of R-spondin 2 and/or R-spondin 3 mediated bone morphogenetic protein (BMP) receptor inhibition for use in treating and/or preventing cancer in a subject.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule or derivative thereof comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fab fragment, but also e.g. a single-chain antibody, a bispecific antibody, and a nanobody are referred to as fragments of an immunoglobulin.

Unless specifically indicated otherwise herein, the compounds specified may be comprised in larger structures, e.g. may be covalently or non-covalently linked to carrier molecules, retardants, and other excipients. In particular, polypeptides as specified may be comprised in fusion polypeptides comprising further peptides, which may serve e.g. as a tag for purification and/or detection, or as a linker. The term “detectable tag” refers to a stretch of amino acids which are added to or introduced into the fusion polypeptide; preferably, the tag is added C- or N-terminally to the fusion polypeptide of the present invention. Said stretch of amino acids preferably allows for detection of the fusion polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants.

The term “R-spondin 2” is known to the skilled person. The human R-spondin 2 polypeptide has several isoforms, the amino acid sequence e.g. of isoform 1 precursor being provided as Genbank Acc No. NP_848660.3, SEQ ID NO:1. In accordance, the term “R-spondin 2”, as used herein, preferably relates to the aforesaid human R-spondin 2, or to a polypeptide having an amino acid sequence at least 70%, preferably at least 80%, more preferably at least 90%, even more preferably at least 95%, still more preferably at least 98%, most preferably at least 99% identical to the amino acid sequence of said human R-spondin 2. Preferably, the R-spondin 2 is human R-spondin 2 or a homolog thereof, preferably a vertebrate homolog, more preferably a mammalian homolog. Homologs of R-spondin 2 are known e.g. from HomoloGene database entry 18235; moreover R-spondin 2 homologues in other species can be identified by sequence comparison, in particular by determining the degree of identity as described elsewhere herein. Thus, the R-spondin 2 preferably is R-spondin 2 of a human, a chimpanzee, a rhesus monkey, a rat, a mouse, a cattle, a dog, a chicken, a zebrafish, or from a western clawed frog, more preferably of a human.

The term “R-spondin 3” is also known to the skilled person. The amino acid sequence of human R-spondin 3 precursor is available e.g. as Genbank Acc. No. NP_116173.2, SEQ ID NO:2. In accordance, the term “R-spondin 3”, as used herein, preferably relates to the aforesaid human R-spondin 3, or to a polypeptide having an amino acid sequence at least 50%, preferably at least 60%, more preferably at least 70%, even more preferably at least 80%, still more preferably at least 90%, most preferably at least 95% identical to the amino acid sequence of said human R-spondin 3. Preferably, the R-spondin 3 is human R-spondin 3 or a homolog thereof, preferably a vertebrate homolog, more preferably a mammalian homolog. Homologs of R-spondin 3 are known e.g. from HomoloGene database entry 12484; moreover R-spondin 3 homologues in other species can be identified by sequence comparison, in particular by determining the degree of identity as described elsewhere herein. Thus, the R-spondin 3 preferably is R-spondin 3 of a human, a chimpanzee, a rhesus monkey, a rat, a mouse, a cattle, a chicken, a zebrafish, or from a western clawed frog, more preferably of a human.

The term “ZNRF3” is known to the skilled person to relate to the E3 ubiquitin-protein ligase known under this designation. The amino acid sequence of human ZNRF3 is available e.g. as Genbank Acc. No. NP_001193927.1, SEQ ID NO:3. In accordance, the term “ZNRF3”, as used herein, preferably relates to the aforesaid human ZNRF3, or to a polypeptide having an amino acid sequence at least 60%, preferably at least 80%, more preferably at least 90%, even more preferably at least 95%, still more preferably at least 98%, most preferably at least 99% identical to the amino acid sequence of said human ZNRF3. Preferably, the ZNRF3 is human ZNRF3 or a homolog thereof, preferably a vertebrate homolog, more preferably a mammalian homolog. Homologs of ZNRF3 are known e.g. from HomoloGene database entry 46592; moreover ZNRF3 homologues in other species can be identified by sequence comparison, in particular by determining the degree of identity as described elsewhere herein. Thus, the ZNRF3 preferably is ZNRF3 of a human, a chimpanzee, a rhesus monkey, a rat, a mouse, a dog, a cattle, a chicken, a zebrafish, or from a western clawed frog, more preferably of a human.

The term “bone morphogenetic protein receptor”, also referred to as “BMP receptor” and as “BMPR”, is also known to the skilled person and relates to a member of the family of transmembrane serine/threonine kinases known under this designation. Preferably, the BMP receptor is BMPR1A, also known as ALK3. The amino acid sequence of human BMPR1A precursor is available e.g. as Genbank Acc No. NP_004320.2, SEQ ID NO:5. In accordance, the term “BMP receptor”, as used herein, preferably relates to the aforesaid human BMPR1A, or to a polypeptide having an amino acid sequence at least 80%, preferably at least 85%, more preferably at least 90%, even more preferably at least 95%, still more preferably at least 98%, most preferably at least 99% identical to the amino acid sequence of said human BMPR1A. Preferably, the BMP receptor is human BMP receptor or a homolog thereof, preferably a vertebrate homolog, more preferably a mammalian homolog. Homologs of BMP receptors are known, e.g. from HomoloGene database entry 20911; moreover, BMP receptor homologues in other species can be identified by sequence comparison, in particular by determining the degree of identity as described elsewhere herein. Thus, the BMP receptor preferably is BMP receptor of a human, a chimpanzee, a rhesus monkey, a rat, a mouse, a dog, a cattle, a chicken, a zebrafish, or from a western clawed frog, more preferably of a human.

The term “BMP receptor inhibition” is, in principle, understood by the skilled person to relate to any modulation causing BMP receptor signaling, preferably BMPR1A signaling, in a host cell to decrease. As is understood by the skilled person, said modulation may be achieved indirectly, e.g. by reducing the amount of BMP receptor ligand, or directly, e.g. by preventing BMP receptor/ligand interaction, or by reducing the amount of BMP receptor present in a cell. Preferably, inhibiting a BMP receptor is reducing the amount of BMP receptor in a cell, more preferably is reducing the amount of BMP receptor present in the cell membrane of a host cell. Thus, preferably, inhibiting a BMP receptor is increasing membrane clearance of a BMP receptor. Methods for determining BMP receptor inhibition are known in the art and are shown herein in the Examples. A preferred method of determining BMP receptor inhibition is determining the amount of BMP receptor in a cell, preferably compared to a control cell. In accordance with the above, a “BMP receptor inhibitor” is a compound mediating BMP receptor inhibition as specified above. As shown in this specification, R-spondin 2 and R-spondin 3 are BMP receptor inhibitors, preferably causing increased membrane clearance of BMPR1A.

In accordance with the above, the term “inhibitor of BMP receptor inhibition”, as used herein, relates to a compound preventing BMP receptor inhibition from occurring in a cell. Thus, the inhibitor of BMP receptor inhibition preferably causes an increase in BMP receptor gene expression, decreases BMP receptor membrane clearance and/or degradation, prevents interaction of the BMP receptor with a BMP receptor inhibitor, and/or decreases the amount of BMP receptor inhibitor in a cell, tissue, bodily fluid, organ, and/or subject. As will be understood, an “inhibitor of R-spondin 2 and/or R-spondin 3 mediated BMP receptor inhibition” is a compound preventing BMP receptor inhibition by R-spondin 2 and/or R-spondin 3 from occurring in a cell. More preferably, the inhibitor of R-spondin 2 and/or R-spondin 3 mediated BMP receptor inhibition decreases R-spondin 2 and/or R-spondin 3 mediated BMP receptor membrane clearance and/or degradation, prevents interaction of the BMP receptor with a R-spondin 2 and/or R-spondin 3, and/or decreases the amount of R-spondin 2 and/or R-spondin 3 in a cell, tissue, bodily fluid, organ, and/or subject. Thus, the inhibitor of R-spondin 2 and/or R-spondin 3 mediated BMP receptor inhibition preferably is selected from the list consisting of an immunoglobulin or fragment thereof, a polypeptide comprising an isolated domain of R-spondin 2 and/or R-spondin 3, a polypeptide comprising an extracellular domain of a BMP receptor, a siRNA, a gRNA, a peptide aptamer, a polynucleotide aptamer, an anticalin, and a Designed Ankyrin Repeat Protein.

Preferably, the inhibitor of R-spondin 2 and/or R-spondin 3 mediated BMP receptor inhibition is an immunoglobulin. As used herein, the term “immunoglobulin” relates to any polypeptide or fragment thereof from the class of polypeptides known to the skilled person under this designation and comprising at least one antigen binding site. Preferably, the immunoglobulin is a soluble immunoglobulin from any of the classes IgA, IgD, IgE, IgG, or IgM, or a fragment comprising at least one antigen binding site derived thereof. Also comprised as immunoglobulins of the present invention are a bispecific immunoglobulin, a synthetic immunoglobulin, an immunoglobulin fragment, such as Fab, Fv or scFv fragments etc., a single chain immunoglobulin, and a nanobody. Further included are chemically modified derivatives of any of the aforesaid, e.g. PEGylated derivatives, as well as fusion proteins comprising any of the aforesaid immunoglobulins and fragments thereof. The immunoglobulin may be a human or humanized immunoglobulin, a primatized, or a chimerized immunoglobulin or a fragment thereof as specified above. Preferably, the immunoglobulin of the present invention is a polyclonal or a monoclonal immunoglobulin, more preferably a monoclonal immunoglobulin or a fragment thereof as specified above. Preferably, the immunoglobulin of the present invention shall specifically bind (i.e. does not cross react with other polypeptides or peptides) to the R-spondin 2 and/or R-spondin 3 and/or to the BMP receptor as specified herein. Specific binding can be tested by various well known techniques.

Methods for preparing antibodies are, in principle, known in the art. Antibodies against target polypeptides can be prepared by well-known methods e.g. using a purified protein or a suitable fragment derived therefrom as an antigen.

Preferably, the inhibitor of R-spondin 2 and/or R-spondin 3 mediated BMP receptor inhibition is an immunoglobulin or fragment thereof as specified herein above specifically binding to R-spondin 2 or R-spondin 3, preferably to R-spondin 2. More preferably, the inhibitor of R-spondin 2 and/or R-spondin 3 mediated BMP receptor inhibition is an immunoglobulin or fragment thereof as specified herein above specifically binding to the TSP1 domain and/or the FU1 domain of said R-spondin, preferably specifically binds the TSP1 domain of said R-spondin. As used herein, the term “TSP1 domain” relates to a fragment of R-spondin 2 or R-spondin 3 corresponding to amino acids 144 to 204 of human R-spondin 2, preferably human R-spondin 2 having the amino acid sequence as shown in Genbank Acc No. NP_848660.3 (SEQ ID NO:5). Whether an amino acid of a polypeptide of interest corresponds to an amino acid as indicated can be established by the skilled person, preferably by performing an alignment of the amino acid sequences of the two polypeptides and determining which amino acid is commensurate in position to the indicated amino acid. Also as used herein, the term “FU1 domain” relates to a fragment of R-spondin 2 or R-spondin 3 corresponding to amino acids 37 to 84 of human R-spondin 2, preferably human R-spondin 2 having the amino acid sequence as shown in Genbank Acc No. NP_848660.3, SEQ ID NO:6.

Preferably, the inhibitor of R-spondin 2 and/or R-spondin 3 mediated BMP receptor inhibition is an immunoglobulin or fragment thereof as specified herein above specifically binding to the extracellular domain of a BMP receptor, preferably of BMP receptor 1A. As used herein, the term “extracellular domain of a BMP receptor” relates to a fragment of a BMP receptor corresponding to amino acids 1 to 152 of the human BMP receptor 1A, preferably human BMP receptor 1A comprising the amino acid sequence as shown in Genbank Acc No. NP_004320.2. Preferably, the inhibitor of R-spondin 2 and/or R-spondin 3 mediated BMP receptor inhibition is an immunoglobulin or fragment thereof as specified herein above specifically binding to the activin receptor domain of the BMP receptor, preferably corresponding to amino acids 59 to 138 of the human BMP receptor 1A, preferably human BMP receptor 1A comprising the amino acid sequence as shown in Genbank Acc No. NP_004320.2, SEQ ID NO:7.

Preferably, the inhibitor of R-spondin 2 and/or R-spondin 3 mediated BMP receptor inhibition is a polypeptide comprising an isolated domain of said R-spondin, preferably a TSP1 domain or an FU1 domain as specified herein above. As used herein, the term “polypeptide comprising an isolated domain of an R-spondin” relates to the property of a polypeptide comprising either a TSP1 domain of an R-spondin or an FU1 domain of an R-spondin, but not both a TSP1 domain and an FU1 domain of an R-spondin. Thus, in case the inhibitor of R-spondin 2 and/or R-spondin 3 mediated BMP receptor inhibition comprises a TSP1 domain, it lacks an FU1 domain, more preferably lacks any domain binding to a ZNRF3 polypeptide; and in case the inhibitor of R-spondin 2 and/or R-spondin 3 mediated BMP receptor inhibition comprises an FU1 domain, it lacks a TSP1 domain, more preferably lacks any domain binding to a BMP receptor. As the skilled person understands, the TSP1 domain comprised in the inhibitor of R-spondin 2 and/or R-spondin 3 mediated BMP receptor inhibition may be a mutated form (mutein) or fragment or other derivative of a TSP1 domain as specified, provided it still binds to the extracellular domain of a BMP receptor, preferably BMPR1A; also, the FU1 domain comprised in the inhibitor of R-spondin 2 and/or R-spondin 3 mediated BMP receptor inhibition may be a mutated form (mutein) or fragment or other derivative of an FU1 domain as specified, provided it still binds to the extracellular domain of a ZNRF3 polypeptide, preferably human ZNRF3 as specified herein above.

Preferably, the inhibitor of R-spondin 2 and/or R-spondin 3 mediated BMP receptor inhibition is a polypeptide comprising an extracellular domain of a BMP receptor, preferably of BMP receptor 1A, more preferably as specified herein above. More preferably, the inhibitor of R-spondin 2 and/or R-spondin 3 mediated BMP receptor inhibition is a polypeptide comprising an amino acid sequence corresponding to amino acids 1 to 152 of the human BMP receptor 1A, preferably human BMP receptor 1A comprising the amino acid sequence as shown in Genbank Acc No. NP_004320.2, SEQ ID NO:6. Still more preferably, the inhibitor of R-spondin 2 and/or R-spondin 3 mediated BMP receptor inhibition is a polypeptide comprising an amino acid sequence corresponding to amino acids 59 to 138 of the human BMP receptor 1A, preferably human BMP receptor 1A comprising the amino acid sequence as shown in Genbank Acc No. NP_004320.2, SEQ ID NO:7.

As the skilled person understands, the extracellular domain of a BMP receptor comprised in the inhibitor of R-spondin 2 and/or R-spondin 3 mediated BMP receptor inhibition may be a mutated form (mutein) or fragment or other derivative of an extracellular domain of a BMP receptor as specified, provided it still binds to the TSP1 domain of an R-spondin 2 and/or R-spondin 3.

Preferably, the inhibitor of R-spondin 2 and/or R-spondin 3 mediated BMP receptor inhibition is an RNAi agent. As used herein, the term “RNAi agent” refers to an shRNA, a siRNA agent, or a miRNA agent as specified below, causing expression of R-spondin 2 and/or R-spondin 3 in a target cell to decrease compared to a control target cell. The RNAi agent is of sufficient length and complementarity to stably interact with the target RNA, i.e. it comprises at least 15, at least 17, at least 19, at least 21, at least 22 nucleotides complementary to the target RNA. By “stably interact” is meant interaction of the RNAi agent or its products produced by the cell with a target RNA, e.g., by forming hydrogen bonds with complementary nucleotides in the target RNA under physiological conditions. As the skilled person understands, the RNAi agent may also be a chemical derivative of a polynucleotide, e.g. a morpholino. Preferably, said morpholino comprises the sequence GCCGTCCAAATGCAGTTTCAAC (SEQ ID NO:9.

The term “siRNA agent” as meant herein encompasses: a) a dsRNA consisting of at least 15, at least 17, at least 19, at least 21 consecutive nucleotides base-paired, i.e. forming hydrogen bonds with complementary nucleotides. b) a small interfering RNA (siRNA) molecule or a molecule comprising an siRNA molecule. The siRNA is a single-stranded RNA molecule with a length, preferably, greater than or equal to 15 nucleotides and, preferably, a length of 15 to 49 nucleotides, more preferably 17 to 30 nucleotides, and most preferably 17 to 30 nucleotides, preferably 17, 18, 19, 20, 21, 22, 23, 24, 25, 26, 27, 28, 29, or 30 nucleotides. According to the present invention, the term “molecule comprising a siRNA molecule” includes RNA molecules from which a siRNA is processed by a cell, preferably by a mammalian cell. Thus, a molecule comprising a siRNA molecule, preferably, is a small hairpin RNA, also known as shRNA. As used herein, the term “shRNA” relates to a, preferably artificial, RNA molecule forming a stem-loop structure comprising at least 10, preferably at least 15, more preferably at least 17, most preferably at least 20 nucleotides base-paired to a complementary sequence on the same mRNA molecule (“stem”), i.e. as a dsRNA, separated by a stretch of non-base-paired nucleotides (“loop”). c) a polynucleotide encoding a) or b), wherein, preferably, said polynucleotide is operatively linked to an expression control sequence. Thus, the function of the siRNA agent to inhibit expression of the target gene can preferably be modulated by said expression control sequence. Preferred expression control sequences are those which can be regulated by exogenous stimuli, e.g. the tet operator, whose activity can be regulated by tetracycline, or heat inducible promoters. Alternatively or in addition, one or more expression control sequences can be used which allow tissue-specific expression of the siRNA agent. siRNAs against the gene encoding e.g. R-spondin 2 are commercially available. The shRNA, preferably comprises the sequence

(SEQ ID NO: 10)
GACAATGGGTGTAGCCGATctcgagATCGGCTACACCCATTGTC.

It is, however, also contemplated that the RNAi agent is a miRNA agent. A “miRNA agent” as meant herein encompasses: a) a pre-microRNA, i.e. a mRNA comprising at least 30, at least 40, at least 50, at least 60, at least 70 nucleotides base-paired to a complementary sequence on the same mRNA molecule (“stem”), i.e. as a dsRNA, separated by a stretch of non-base-paired nucleotides (“loop”). b) a pre-microRNA, i.e. a dsRNA molecule comprising a stretch of at least 19, at least 20, at least 21, at least 22, at least 23, at least 24, at least 25 base-paired nucleotides formed by nucleotides of the same RNA molecule (stem), separated by a loop. c) a microRNA (miRNA), i.e. a dsRNA comprising at least 15, at least 17, at least 18, at least 19, at least 21 nucleotides on two separate RNA strands. d) a polynucleotide encoding a) or b), wherein, preferably, said polynucleotide is operatively linked to an expression control sequence as specified above.

Also preferably, the inhibitor of R-spondin 2 and/or R-spondin 3 mediated BMP receptor inhibition comprises at least one, preferably two, gRNAs, i.e. preferably CRISPR/Cas targeting oligonucleotides, targeting the gene encoding R-spondin 2 and/or R-spondin 3, preferably R-spondin 2. The CRISPR/Cas system has been known for several years as a convenient system for inducing knock-out mutations, i.e. deletions, preferably of chromosomal genes. The skilled person knows how to design appropriate oligonucleotides, which are, preferably, expressed from a vector, to induce deletion of a DNA sequence of interest. Preferably, said deletion is a partial deletion, more preferably deletion of a portion of the gene essential for function; most preferably said deletion is a complete deletion of at least the whole coding region. Preferably, the R-spondin 2 gRNA comprises the sequence TGACTCCATAGTATCCAGGA (SEQ ID NO:11).

Preferably, the inhibitor of R-spondin 2 and/or R-spondin 3 mediated BMP receptor inhibition is an aptamer. As used herein, the term “aptamer” relates to a polynucleotide or polypeptide binding specifically to a target molecule by virtue of its three-dimensional structure. Preferably, the aptamer specifically interacts with an R-spondin 2 and/or R-spondin 3 or with a BMP receptor as specified for the immunoglobulins described herein above. Preferably, the aptamer is a peptide aptamer. Peptide aptamers, preferably, are peptides comprising 8-80 amino acids, more preferably 10-50 amino acids, and most preferably 15-30 amino acids. They can e.g. be isolated from randomized peptide expression libraries in a suitable host system like baker's yeast (see, for example, Klevenz et al., Cell Mol Life Sci. 2002, 59:1993-1998). A peptide aptamer, preferably, is a free peptide; it is, however, also contemplated that a peptide aptamer is fused to a polypeptide serving as “scaffold”, meaning that the covalent linking to said polypeptide serves to fix the three-dimensional structure of said peptide aptamer to a specific conformation.

Preferably, the inhibitor of R-spondin 2 and/or R-spondin 3 mediated BMP receptor inhibition is an anticalin. As used herein, the term “anticalin” relates to an artificial polypeptide derived from a lipocalin specifically binding an R-spondin 2 and/or R-spondin 3 or a BMP receptor as specified for the immunoglobulins described herein above. Similarly, a “Designed Ankyrin Repeat Protein” or “DARPin”, as used herein, is an artificial polypeptide, comprising several ankyrin repeat motifs, specifically binding an R-spondin 2 and/or R-spondin 3 or a BMP receptor as specified for the immunoglobulins described herein above.

Preferably, the inhibitor of R-spondin 2 and/or R-spondin 3 mediated BMP receptor inhibition is retinoic acid, preferably all-trans retinoic acid ((2E,4E,6E,8E)-3,7-dimethyl-9-(2,6,6-trimethylcyclohexen-1-yl) nona-2,4,6,8-tetraenoic acid, CAS No: 302-79-4).

Preferably, the inhibitor of R-spondin 2 and/or R-spondin 3 mediated BMP receptor inhibition is comprised in a pharmaceutical composition, said pharmaceutical composition preferably further comprising a pharmaceutically acceptable carrier. The term “pharmaceutical composition”, as used herein, thus relates to a composition comprising the inhibitor of R-spondin 2 and/or R-spondin 3 mediated BMP receptor inhibition in a pharmaceutically acceptable form and, optionally, a pharmaceutically acceptable carrier. The compounds of the present invention can be formulated as pharmaceutically acceptable salts. Acceptable salts comprise acetate, methylester, HCl, sulfate, chloride and the like. The pharmaceutical compositions are, preferably, administered topically or systemically. Suitable routes of administration conventionally used for drug administration are oral, intravenous, or parenteral administration as well as inhalation. Preferably, the pharmaceutical composition of the present invention is administered via a parenteral route, preferably by intravenous injection. However, polynucleotide compounds may also be administered in a gene therapy approach by using viral vectors, viruses or liposomes, and may also be administered topically, e.g. as an ointment. Moreover, the compounds can be administered in combination with other drugs either in a common pharmaceutical composition or as separated pharmaceutical compositions wherein said separated pharmaceutical compositions may be provided in form of a kit of parts. The compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables. The carrier(s) must be acceptable in the sense of being compatible with the other ingredients of the formulation and being not deleterious to the recipient thereof. The pharmaceutical carrier employed may be, for example, either a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania.

The diluent(s) is/are preferably selected so as not to affect the biological activity of the inhibitor of R-spondin 2 and/or R-spondin 3 mediated BMP receptor inhibition and potential further pharmaceutically active ingredients. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the compounds to be used in a pharmaceutical composition of the present invention which prevents, ameliorates or treats a condition referred to herein. Therapeutic efficacy and toxicity of compounds can be determined by standard pharmaceutical procedures in cell culture or in experimental animals, e.g., by determining the ED50 (the dose therapeutically effective in 50% of the population) and/or the LD50 (the dose lethal to 50% of the population). The dose ratio between therapeutic and toxic effects is the therapeutic index, and it can be expressed as the ratio, LD50/ED50. The dosage regimen will be determined by the attending physician, preferably taking into account relevant clinical factors and, preferably, in accordance with any one of the methods described elsewhere herein. As is well known in the medical arts, a dosage for any one patient may depend upon many factors, including the patient's size, body surface area, age, the particular compound to be administered, sex, time and route of administration, general health, and other drugs being administered concurrently. Progress can be monitored by periodic assessment. A typical dose can be, for example, in the range of 1 μg to 10000 μg; however, doses below or above this exemplary range are envisioned, especially considering the aforementioned factors. The pharmaceutical compositions and formulations referred to herein are administered at least once in order to treat or prevent a disease or condition recited in this specification. However, the said pharmaceutical compositions may be administered more than one time, for example, preferably from one to four times, more preferably two or three times.

In a preferred embodiment, the pharmaceutical composition comprises at least one further pharmaceutically active compound, i.e. preferably is a combined preparation. The term “combined preparation” as referred to in this application preferably comprises all pharmaceutically active compounds in one preparation so that all compounds are administered simultaneously and in the same way. Also preferably, the combined preparation comprises at least two physically separated preparations for separate administration, wherein each preparation contains at least one pharmaceutically active compound. The latter alternative is preferred in cases where the pharmaceutically active compounds of the combined preparation have to be administered by different routes, e.g. parenterally and orally, due to their chemical or physiological properties. Preferably, the at least two separated preparations are administered simultaneously. This means that the time frames of the administration of the preparations overlap. Also preferred is the sequential administration of the at least two preparations, whereas the administration of the single preparations shall occur in time frames which do not overlap so that the at least to pharmaceutically active compounds of the preparations are present in such plasma concentrations which enable the synergistic effect of the present invention. Preferably, the at least two preparations are administered in a time interval of 1 minute, 5 minutes, 15 minutes, 30 minutes, 1 hour, 2 hours, 4 hours, 8 hours, 16 hours, 1 day or 2 days, preferably within 1 hour, more preferably simultaneously, most preferably in a combined preparation comprising all pharmaceutically active compounds.

In a preferred embodiment, the further pharmaceutically active compound in the combined preparation is an antiproliferative agent, more preferably a chemotherapeutic agent. These substances lead to cell damage, e.g. to the DNA. Antiproliferative and chemotherapeutic compounds are known to the person skilled in the art. Preferred chemotherapeutic agents are selected from the group consisting of antimetabolites, Bleomycins, DNA-crosslinking agents, Anthracyclines, topoisomerase poisons, monoclonal antibodies, biological response modifiers, tyrosine kinase inhibitors, aromatase inhibitors, aurora kinase inhibitors, histone deacetylase inhibitors, metalloprotease inhibitors, RAS-MAPK inhibitors, enzymes and spindle poisons. More preferably, the chemotherapeutic agent is an antimetabolite, preferably selected from cytarabine, methotrexate, 6-mercaptopurine, fludarabine, cladribine, 5-fluorouracil, capecitabine, gemcitabine and hydroxyurea. An especially preferred chemotherapeutic agent is cytarabine.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating leukemia is reducing leukemia cell count in a subject, preferably leukemia cell count in blood. As will be understood by the skilled person, effectiveness of treatment of e.g. leukemia is dependent on a variety of factors including, e.g. cancer stage and cancer type. Thus, preferably, treating has the effect of causing leukemia cells to stop growing, more preferably causing leukemia to resolve.

The term “preventing” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein, in particular a leukemia relapse. Whether a portion is statistically significant can be determined without further ado by the person skilled in the art using various well known statistic evaluation tools discussed elsewhere in this specification.

In a preferred embodiment, treating and/or preventing comprises co-administration of an antiproliferative agent, more preferably a chemotherapeutic agent, as specified herein above. Thus, the present invention also relates to an inhibitor of R-spondin 2 and/or R-spondin 3 mediated bone morphogenetic protein (BMP) receptor inhibition for use in treating and/or preventing leukemia in a subject with an antiproliferative agent; and relates to an antiproliferative agent for use in treating and/or preventing leukemia in a subject with an inhibitor of R-spondin 2 and/or R-spondin 3 mediated bone morphogenetic protein (BMP) receptor inhibition.

“Cancer” in the context of this invention refers to a disease of an animal, including man, characterized by uncontrolled growth by a group of body cells (“cancer cells”). This uncontrolled growth may be accompanied by spread of cancer cells to other locations in the body, e.g. via the blood system, e.g. as metastases, or by the spread of cancer cells e.g. in non-solid cancers such as leukemia. Moreover, cancer may entail recurrence of cancer cells after an initial treatment apparently removing cancer cells from a subject (“relapse”). Preferably, the cancer is leukemia or colon cancer, in particular colorectal cancer. More preferably, the cancer is leukemia. The term “leukemia” is known to the skilled person. Preferably, the term includes all types of blood cancers referred to under this designation, including acute and chronic leukemias, as well as myeloid and lymphoid leukemias. Preferably, leukemia is a myeloid leukemia, i.e. a cancer of the myeloid line of blood cells, more preferably is monocytic leukemia. Also preferably leukemia is acute leukemia. More preferably, leukemia is acute myeloid leukemia, most preferably is acute monocytic leukemia (AML).

The term “subject”, as used herein, relates to a vertebrate organism, preferably a mammal, even more preferably a livestock or companion animal, such as a chicken, a goose, a duck, a goat, a sheep, a cattle, a pig, a horse, a dog, a cat, a hamster, a rat, a mouse, a hamster, or a guinea pig. Most preferably, the subject is a human. Preferably, the subject is known or suspected to suffer or have suffered from leukemia as specified herein above. Also preferably, the subject was identified to benefit from leukemia treatment with an inhibitor of R-spondin 2 or R-spondin 3 mediated BMP receptor inhibition, preferably according to the method as specified herein below, and/or was identified as a subject suffering from a severe form of leukemia according to the method as specified herein below. Thus, preferably, said subject is suffering from a leukemia in which leukemia cells comprise a decreased activity of a BMP receptor, preferably BMPR1A, preferably caused by R-spondin 2 or R-spondin 3 overproduction.

The term “host cell”, preferably, relates to vertebrate cell, preferably a mammalian cell, more preferably a human cell. Preferably, the host cell is a cell of a subject as specified herein above. Preferably, the host cell is an in vivo cell, i.e. a cell comprised in a living subject. More preferably, the host cell is a cell maintained in vitro, preferably in a suitable cultivation medium. Preferably, the host cell is a cell of a leukemia as specified herein above.

Advantageously, it was found in the work underlying the present invention that leukemia cells may overproduce R-spondin 2 and/or 3, causing a decrease in BMP receptor activity, in turn leading to lack of differentiation of leukemia cells and increase of proliferation. Thus, compounds preventing the aforesaid decrease in BMP receptor activity are suitable for promoting differentiation of leukemia cells and decreasing their proliferation. Moreover, it was found that the extent of decrease in BMP receptor activity, e.g. measured by the degree of R-spondin 2 and/or 3 overproduction, is a predictor of prognosis, in particular in AML patients.

The definitions made above apply mutatis mutandis to the following. Additional definitions and explanations made further below also apply for all embodiments described in this specification mutatis mutandis.

The present invention further relates to a method for identifying a subject benefiting from leukemia treatment with an inhibitor of R-spondin 2 and/or R-spondin 3 mediated BMP receptor inhibition comprising
(a) contacting a sample comprising leukemia cells of said subject with an inhibitor of R-spondin 2 and/or R-spondin 3 mediated BMP receptor inhibition;
(b) determining the amount of BMP receptor, phospho-mothers against decapentaplegic homolog 1 (pSMAD1), DNA-binding protein inhibitor ID-1 (ID1), CD14, and/or integrin alpha-M (CD11B) in the leukemia cells of step (a),
(c) comparing the amount determined in step (b) to a reference, preferably from control treated leukemia cells of said subject, and
(d) based on the result of step (c), identifying a subject benefiting from treatment with an inhibitor of R-spondin 2 and/or R-spondin 3 mediated BMP receptor inhibition.

The method for identifying a subject benefiting from leukemia treatment with an inhibitor of R-spondin 2 and/or R-spondin 3 mediated BMP receptor inhibition of the present invention, preferably, is an in vitro method. Moreover, it may comprise steps in addition to those explicitly mentioned above. For example, further steps may relate, e.g., to providing a sample for step a), or determining additional markers in step b). Moreover, one or more of said steps may be performed by automated equipment. Also, the method for identifying a subject benefiting from leukemia treatment with an inhibitor of R-spondin 2 and/or R-spondin 3 mediated BMP receptor inhibition may be part of a method of treatment comprising first identifying a subject benefiting from leukemia treatment with an inhibitor of R-spondin 2 and/or R-spondin 3 mediated BMP receptor inhibition and then treating said subject with an inhibitor of R-spondin 2 and/or R-spondin 3 mediated BMP receptor inhibition, preferably as specified herein below.

The term “contacting” is understood by the skilled person. Preferably, the term relates to bringing a compound as specified into physical contact with a sample or with a further compound and thereby, e.g. allowing the sample and the compound to interact.

The term “sample”, as used herein, relates to a sample of a body fluid, a sample from a tissue or an organ, or a sample of wash/rinse fluid or a swab or smear obtained from an outer or inner body surface, said sample being known or suspected to comprise leukemia cells. Preferably, the sample is a blood, plasma, serum, urine, saliva, or lacrimal fluid sample. Samples can be obtained by use of brushes, (cotton) swabs, spatula, rinse/wash fluids, punch biopsy devices, puncture of cavities with needles or lancets, or by surgical instrumentation. However, samples obtained by well-known techniques including, in an embodiment, scrapes, swabs or biopsies from the urogenital tract, perianal regions, anal canal, the oral cavity, the upper aerodigestive tract and the epidermis are also included. Preferably, samples are obtained from body fluids known to comprise leukemia cells if present in a subject, i.e., preferably, blood, saliva, or bone marrow aspirate, more preferably blood.

It is to be understood that the sample may be further processed in order to carry out the method of the present invention. Particularly, cells may be obtained from the sample by methods and means known in the art. Thus, the term sample also may relate to preparations comprising or suspected to comprise leukemia cells, diluted, enriched, purified and/or cultivated from a sample.

The term “mothers against decapentaplegic homolog 1”, also referred to as “SMAD1”, is known to the skilled person. The amino acid sequence of human SMAD1 is available e.g. from Genbank Acc. No. AAC50790.1. pSMAD relates to a form of the SMAD1 polypeptide which is phosphorylated at amino acid S463 and/or S465.

The term “DNA-binding protein inhibitor ID-1”, also referred to as “ID-1”, is also known to the skilled person. The amino acid sequence of human ID-1 is available e.g. from Genbank Acc. No. NP_851998.1.

The term “monocyte differentiation antigen CD14”, also referred to as “CD14” is known to the skilled person as well. The amino acid sequence of human CD14 is available e.g. from Genbank Acc. No. NP_001167576.1.

the skilled person also knows “integrin alpha-M”, also referred to as “CD11B”. The amino acid sequence of the precursor of isoform 1 of human CD141B is available e.g. from Genbank Acc. No. NP_001139280.1

Methods for determining the amounts of the aforesaid markers are known in the art. The skilled person is aware that in principle the amount of RNA expressed by a cell for a marker can be determined, except for pSMAD1; however, preferably, the amount of the indicated polypeptide(s) in the cells is determined, more preferably by an immunoassay.

Also, the skilled person is able to provide appropriate references. The reference may be a threshold vale, a reference range, a score including the amount(s) of the marker(s) determined as a parameter, or any other reference deemed appropriate by the skilled person. For establishing a score, other parameters may additionally be included as parameters, e.g. leukemia stage, histological parameters such as degree of differentiation of leukemia cells, risk factors associated with the subject, and/or results of genetic assessments, e.g. evaluation of chromosome aberrations in leukemia cells of the subject. Preferably, the reference is derived from a population of apparently healthy subjects or from a population of subjects known not to benefit from treatment with an inhibitor of R-spondin 2 and/or R-spondin 3 mediated BMP receptor inhibition; if, in such case, a threshold value is defined based on said population, a subject benefiting from treatment with an inhibitor of R-spondin 2 and/or R-spondin 3 mediated BMP receptor inhibition is preferably identified if the amount determined in step (b) is lower than the reference. Also preferably, the reference is derived from a population of subjects known to benefit from treatment with an inhibitor of R-spondin 2 and/or R-spondin 3 mediated BMP receptor inhibition; if, in such case, a threshold value is defined based on said population, a subject benefiting from treatment with an inhibitor of R-spondin 2 and/or R-spondin 3 mediated BMP receptor inhibition is identified if the amount determined in step (a) is equal to or higher than the reference. Also preferably, the reference is derived from control treated leukemia cells of said subject, i.e., preferably, leukemia cells of said subject not treated with an inhibitor of R-spondin 2 and/or R-spondin 3 mediated BMP receptor inhibition, but, more preferably, otherwise treated in an identical manner; if, in such case, a threshold value is defined based on said control treated leukemia cells, a subject benefiting from treatment with an inhibitor of R-spondin 2 and/or R-spondin 3 mediated BMP receptor inhibition is identified if the amount determined in step (b) is higher than said reference.

The present invention also relates to a method for identifying a subject suffering from a severe form of leukemia comprising
(a) determining the amount of R-spondin 2, R-spondin 3, and/or a BMP receptor in a sample of said subject,
(b) comparing the amount determined in step (a) to a reference, and
(c) based on the result of step (b), identifying a subject suffering from a severe form of leukemia.

The method for identifying a subject suffering from a severe form of leukemia, preferably, is an in vitro method. Moreover, it may comprise steps in addition to those explicitly mentioned above. For example, further steps may relate, e.g., to providing a sample for step a), and/or determining further parameters indicative of severity of disease before step c). Moreover, one or more of said steps may be performed by automated equipment. Also, the method for identifying a subject suffering from a severe form of leukemia may be part of a method of treatment comprising first identifying a suffering from a severe form of leukemia and then treating said subject with an inhibitor of R-spondin 2 and/or R-spondin 3 mediated BMP receptor inhibition, preferably as specified herein below.

The term “severe form of leukemia”, as used herein, relates to a form of leukemia as specified herein above in which the 3-year survival rate is less than 25%. In accordance, a mild form of leukemia is a form of leukemia in which the 3-year survival rate is at least 25%, preferably at least 35%, more preferably at least 40%. Preferably, in a severe form of leukemia, the leukemia cells overproduce R-spondin 2 and/or R-spondin 3, and comprise a decreased amount of BMP receptor, preferably BMPR1A. As the skilled person understands, it is also possible to identify a subject suffering from a severe form of leukemia by determining at least one of the markers described herein in the context of the method for identifying a subject benefiting from leukemia treatment with an inhibitor of R-spondin 2 and/or R-spondin 3 mediated BMP receptor inhibition described herein above. Thus, preferably, at least one of phospho-mothers against decapentaplegic homolog 1 (pSMAD1), DNA-binding protein inhibitor ID-1 (ID1), CD14, and integrin alpha-M (CD11B). Preferably, however, R-spondin 2, R-spondin 3, and/or a BMP receptor are determined. More preferably, at least one of pSMAD1, ID1, CD14, and CD11B is determined in addition to R-spondin 2, R-spondin 3, and/or a BMP receptor.

For references, reference may be made to the description above relating to references for identifying a subject benefiting from leukemia treatment with an inhibitor of R-spondin 2 and/or R-spondin 3 mediated BMP receptor inhibition, which applies to the method for identifying a subject suffering from a severe form of leukemia mutatis mutandis. Thus, the skilled person is able to establish and use appropriate references. Preferably, the reference is the median of amounts of said R-spondin 2, R-spondin 3, and/or BMP receptor in a population of apparently healthy subjects or in a population of subjects suffering from leukemia, more preferably is the median of amounts of said R-spondin 2, R-spondin 3, and/or BMP receptor in a population of subjects suffering from leukemia; in such case, a subject suffering from a severe form of leukemia is preferably identified if the value determined for said R-spondin 2 and/or R-spondin 3 in step (a) is higher than the reference; and/or if the value determined for said BMP receptor in step (a) is lower than the reference.

The present invention also relates to a method for identifying a compound for treating and/or preventing leukemia, preferably acute myeloid leukemia (AML), comprising
(A) contacting leukemia cells, preferably AML cells, with a compound suspected to be a compound for treating leukemia,
(B) determining an amount of a BMP receptor, phospho-mothers against decapentaplegic homolog 1 (pSMAD1), DNA-binding protein inhibitor ID-1 (ID1), CD14, integrin alpha-M (CD11B), R-spondin 2, and/or R-spondin 3 in said leukemia cells, and
(C) based on the result of step (B), identifying a compound for treating and/or preventing leukemia.

The method for identifying a compound for treating and/or preventing leukemia is an in vitro method. Moreover, it may comprise steps in addition to the mentioned above. Moreover, the method may be assisted or performed in part or as a whole by automated equipment. In particular, it is envisaged that the method is performed using a high-throughput setting.

The term “compound suspected to be a compound for treating leukemia”, also referred to as “candidate compound”, is a broad term including all chemical compounds for which the skilled person may assume that they could be effective in treating and/or preventing leukemia. Preferably, said candidate compound is a macromolecule, preferably from a group of macromolecules as specified herein above. More preferably, the candidate compound is a small molecule compound, preferably with a molecular mass of less than 1000 Da, more preferably less than 750 Da. Preferably, the candidate compound is comprised in a compound library.

In the context of the method for identifying a compound for treating and/or preventing leukemia, the leukemia cells, preferably, are cultured leukemia cells, in particular a leukemia cell line. In such case, a variant of BMP receptor, ID1, CD14, CD11B, R-spondin 2, and/or R-spondin 3 may be expressed in said leukemia cells, said variant comprising e.g. a detectable tag as specified elsewhere herein, to simplify determining the amount of the respective marker. The leukemia cells may, however, also be leukemia cells from a sample of a subject, in which case, preferably, the method is for identifying a compound for treating and/or preventing leukemia to which said leukemia cells are particularly sensitive. In particular, such leukemia cells from a sample of a subject may be cells from a relapse after an initially successful treatment.

The identification step is performed mutatis mutandis compared to the identification steps of the methods referred to herein above. Thus, preferably, a compound for treating and/or preventing leukemia is identified if it induces a decrease of R-spondin 2, and/or R-spondin 3 and/or an increase in BMP receptor, pSMAD1, ID1, CD14, and/or CD11B as compared to control-treated cells.

The present invention also relates to a kit comprising (i) an inhibitor of R-spondin 2 and/or R-spondin 3 and (ii) a means for determining a BMP receptor, phospho-mothers against decapentaplegic homolog 1 (SMAD1), DNA-binding protein inhibitor ID-1 (ID1), CD14, integrin alpha-M (CD11B), R-spondin 2, and/or R-spondin 3 in a sample.

The term “kit”, as used herein, refers to a collection of the aforementioned compounds, means or reagents which may or may not be packaged together. The components of the kit are preferably comprised by separate vials (i.e. as a kit of separate parts). Moreover, it is to be understood that the kit of the present invention, preferably, is to be used for practicing the methods referred to herein above, preferably the method for identifying a subject benefiting from leukemia treatment with an inhibitor of R-spondin 2 and/or R-spondin 3 mediated BMP receptor inhibition. It is, preferably, envisaged that all components are provided in a ready-to-use manner for practicing the methods referred to above. Further, the kit, preferably, contains instructions for carrying out said methods. The instructions can be provided by a user's manual in paper or electronic form. In addition, the manual may comprise instructions for administration and/or dosage instructions for carrying out the aforementioned methods using the kit of the present invention.

The present invention further relates to a method, preferably an in vivo method, of treating and/or preventing leukemia in a subject comprising
(I) contacting said subject with an inhibitor of R-spondin 2 and/or R-spondin 3 mediated BMP receptor inhibition; and
(II) thereby treating and/or preventing leukemia in said subject.

The present invention also relates to a combined preparation comprising (i) an inhibitor of R-spondin 2 and/or R-spondin 3 mediated BMP receptor inhibition or prodrug thereof and (ii) an anticancer agent and/or an immunomodulatory agent; for use in treating and/or preventing leukemia.

The term “combined preparation”, as referred to in this application, relates to a preparation comprising the pharmaceutically active compounds of the present invention in one preparation. Preferably, the combined preparation is comprised in a container, i.e. preferably, said container comprises all pharmaceutically active compounds of the present invention. Preferably, said container comprises the pharmaceutically active compounds of the present invention as separate formulations, i.e. preferably, one formulation of the inhibitor of R-spondin 2 and/or R-spondin 3 mediated BMP receptor inhibition or prodrug thereof and one formulation of anticancer agent and/or immunomodulatory agent. As will be understood by the skilled person, the term “formulation” relates to a, preferably pharmaceutically acceptable, mixture of compounds, comprising or consisting of at least one pharmaceutically active compound of the present invention. Preferably, the combined preparation comprises the inhibitor of R-spondin 2 and/or R-spondin 3 mediated BMP receptor inhibition or prodrug thereof and the anticancer agent and/or immunomodulatory agent in a single solid pharmaceutical form, e.g. a tablet; more preferably, the compounds are comprised in two separate, preferably liquid, formulations; said separate liquid formulations, preferably are for injection.

Preferably, the combined preparation is for separate or for combined administration. “Separate administration”, as used herein, relates to an administration wherein at least two of the pharmaceutically active compounds of the present invention are administered via different routes and/or at different parts of the body of a subject. E.g. one compound may be administered by enteral administration (e.g. orally), whereas a second compound is administered by parenteral administration (e.g. intravenously). Preferably, the combined preparation for separate administration comprises at least two physically separated preparations for separate administration, wherein each preparation contains at least one pharmaceutically active compound; said alternative is preferred e.g. in cases where the pharmaceutically active compounds of the combined preparation have to be administered by different routes, e.g. parenterally and orally, due to their chemical or physiological properties. Conversely, “combined administration” relates to an administration wherein the pharmaceutically active compounds of the present invention are administered via the same route, e.g. orally or, preferably, intravenously.

Also preferably, the combined preparation is for simultaneous or for sequential administration. “Simultaneous administration”, as used herein, relates to an administration wherein the pharmaceutically active compounds of the present invention are administered at the same time, i.e., preferably, administration of the pharmaceutically active compounds starts within a time interval of less than 15 minutes, more preferably, within a time interval of less than 5 minutes. Most preferably, administration of the pharmaceutically active compounds starts at the same time, e.g. by swallowing a tablet comprising the pharmaceutically active compounds, or by swallowing a tablet comprising one of the pharmaceutically active compounds and simultaneous injection of the second compound, or by applying an intravenous injection of a solution comprising one pharmaceutically active compound and injecting second compound in different part of the body. Conversely, “sequential administration, as used herein, relates to an administration causing plasma concentrations of the pharmaceutically active compounds in a subject enabling the synergistic effect of the present invention, but which, preferably, is not a simultaneous administration as specified herein above. Preferably, sequential administration is an administration wherein administration of the pharmaceutically active compounds, preferably all pharmaceutically active compounds, starts within a time interval of 1 or 2 days, more preferably within a time interval of 12 hours, still more preferably within a time interval of 4 hours, even more preferably within a time interval of one hour, most preferably within a time interval of 5 minutes.

The present invention also relates to a use of an inhibitor of R-spondin 2 and/or R-spondin 3 mediated BMP receptor inhibition in the manufacture of a pharmaceutical composition for treating and/or preventing leukemia.

In view of the above, the following embodiments are particularly envisaged:
Embodiment 1. An inhibitor of R-spondin 2 and/or R-spondin 3 mediated bone morphogenetic protein (BMP) receptor inhibition for use in treating and/or preventing leukemia in a subject.
Embodiment 2. The inhibitor for use of embodiment 1, wherein said inhibitor is a high-molecular weight inhibitor with a molecular weight of at least 1 kD, preferably at least 5 kD.
Embodiment 3. The inhibitor for use of embodiment 1 or 2, wherein said inhibitor is selected from the list consisting of an immunoglobulin or binding fragment thereof, a polypeptide comprising an isolated domain of said R-spondin, a polypeptide comprising an extracellular domain of a BMP receptor, an RNAi agent, a gRNA, a peptide aptamer, a polynucleotide aptamer, an anticalin, and a Designed Ankyrin Repeat Protein.
Embodiment 4. The inhibitor for use of any one of embodiments 1 to 3, wherein said inhibitor is selected from
   (i) an immunoglobulin or fragment thereof specifically binding to said R-spondin 2 or R-spondin 3, preferably to said R-spondin 2;
   (ii) an immunoglobulin or fragment thereof specifically binding to the extracellular domain of a BMP receptor, preferably BMP receptor 1A;
   (iii) a polypeptide comprising either the thrombospondin type-1 (TSP1) domain or the first furin-like (FU1) domain of said R-spondin, or a fragment thereof;
   (iv) a polypeptide comprising an extracellular domain of a BMP receptor, preferably of BMP receptor 1A;
   (v) any combination of (i) to (iv).
Embodiment 5. The inhibitor for use of embodiment 4, wherein said immunoglobulin or fragment thereof specifically binding to said R-spondin 2 or R-spondin 3 specifically binds the TSP1 domain and/or the FU1 domain of said R-spondin, preferably specifically binds the TSP1 domain of said R-spondin.
Embodiment 6. The inhibitor for use of embodiment 4 or 5, wherein said TSP1 domain corresponds to amino acids 144 to 204 of a human R-spondin 2, and/or wherein said FU1 domain corresponds to amino acids 37 to 84 of a human R-spondin 2.
Embodiment The inhibitor for use of embodiment 4, wherein said immunoglobulin or subdomain thereof specifically binding to the extracellular domain of a BMP receptor specifically binds the activin receptor domain of said BMP receptor, preferably binds an epitope comprised in a peptide corresponding to amino acids 1 to 152 of the human BMP receptor 1A.
Embodiment 8. The inhibitor for use of any one of embodiments embodiment 4 to 7, wherein said wherein said immunoglobulin is a monoclonal immunoglobulin.
Embodiment 9. The inhibitor for use of any one of embodiments 1 to 8, wherein said subject was identified as benefiting from treatment with an inhibitor of R-spondin 2 or R-spondin 3 according to the method according to any one of embodiments 11 to 13; and/or wherein said subject is suffering from a leukemia in which leukemia cells comprise a decreased activity of said BMP receptor, preferably caused by R-spondin 2 or R-spondin 3 overproduction.
Embodiment 10. The inhibitor for use of embodiment 9, wherein said inhibitor is retinoic acid, preferably all-trans retinoic acid.
Embodiment 11. A method for identifying a subject benefiting from leukemia treatment with an inhibitor of R-spondin 2 and/or R-spondin 3 mediated BMP receptor inhibition comprising
   (a) contacting a sample comprising leukemia cells of said subject with an inhibitor of R-spondin 2 and/or R-spondin 3 mediated BMP receptor inhibition;
   (b) determining the amount of BMP receptor, phospho-mothers against decapentaplegic homolog 1 (pSMAD1), DNA-binding protein inhibitor ID-1 (ID1), CD14, and/or integrin alpha-M (CD11B) in the leukemia cells of step (a),
   (c) comparing the amount determined in step (b) to a reference, preferably from control treated leukemia cells of said subject, and
   (d) based on the result of step (c), identifying a subject benefiting from treatment with an inhibitor of R-spondin 2 and/or R-spondin 3 mediated BMP receptor inhibition.
Embodiment 12. The method of embodiment 11, wherein said reference is derived from a population of apparently healthy subjects or from a population of subjects known not to benefit from treatment with an inhibitor of R-spondin 2 and/or R-spondin 3 mediated BMP receptor inhibition; and wherein a subject benefiting from treatment with an inhibitor of R-spondin 2 and/or R-spondin 3 mediated BMP receptor inhibition is identified if the amount determined in step (b) is higher than the reference; and/or wherein said reference is derived from a population of subjects known to benefit from treatment with an inhibitor of R-spondin 2 and/or R-spondin 3 mediated BMP receptor inhibition; and wherein a subject benefiting from treatment with an inhibitor of R-spondin 2 or R-spondin 3 mediated BMP receptor inhibition is identified if the amount determined in step (a) is equal to or higher than the reference.
Embodiment 13. The method of embodiment 11 or 12, wherein said reference is derived from control treated leukemia cells of said subject and wherein a subject benefiting from treatment with an inhibitor of R-spondin 2 and/or R-spondin 3 mediated BMP receptor inhibition is identified if the amount determined in step (b) is higher than said reference.
Embodiment 14. A method for identifying a subject suffering from a severe form of leukemia comprising
   (a) determining the amount of R-spondin 2, R-spondin 3, and/or a BMP receptor in a sample of said subject,
   (b) comparing the amount determined in step (a) to a reference, and
   (c) based on the result of step (b), identifying a subject suffering from a severe form of leukemia.
Embodiment 15. The method of embodiment 14, wherein said reference is the median of amounts of said R-spondin 2, R-spondin 3, and/or BMP receptor in a population of apparently healthy subjects or in a population of subjects suffering from leukemia; and wherein a subject suffering from a severe form of leukemia is identified if the value determined for said R-spondin 2 and/or R-spondin 3 in step (a) is higher than the reference; and/or if the value determined for said BMP receptor in step (a) is lower than the reference.
Embodiment 16. The subject matter of any one of the preceding embodiments, wherein said subject is a mammal, preferably a human.
Embodiment 17. A method for identifying a compound for treating and/or preventing leukemia, preferably acute myeloid leukemia (AML), comprising
   (A) contacting leukemia cells, preferably AML cells, with a compound suspected to be a compound for treating leukemia,
   (B) determining an amount of a BMP receptor, phospho-mothers against decapentaplegic homolog 1 (SMAD1), DNA-binding protein inhibitor ID-1 (ID1), CD14, integrin alpha-M (CD11B), R-spondin 2, and/or R-spondin 3 in said leukemia cells, and
   (C) based on the result of step (B), identifying a compound for treating and/or preventing leukemia.
Embodiment 18. The method of embodiment 17, wherein in step (B) the amount of a BMP receptor, phospho-mothers against decapentaplegic homolog 1 (SMAD1), DNA-binding protein inhibitor ID-1 (ID1), CD14, and/or integrin alpha-M (CD11B) is determined.
Embodiment 19. A kit comprising (i) an inhibitor of R-spondin 2 and/or R-spondin 3 and (ii) a means for determining a BMP receptor, phospho-mothers against decapentaplegic homolog 1 (SMAD1), DNA-binding protein inhibitor ID-1 (ID1), CD14, integrin alpha-M (CD11B), R-spondin 2, and/or R-spondin 3 in a sample.
Embodiment 20. The subject matter of any one of the preceding embodiments, wherein said R-spondin is human R-spondin, and/or wherein said BMP receptor is a human BMP receptor, preferably human BMP receptor 1A.
Embodiment 21. A method of treating and/or preventing leukemia in a subject comprising
   (I) contacting said subject with an inhibitor of R-spondin 2 and/or R-spondin 3 mediated BMP receptor inhibition; and
   (II) thereby treating and/or preventing leukemia in said subject.
Embodiment 22. A combined preparation comprising (i) an inhibitor of R-spondin 2 and/or R-spondin 3 mediated BMP receptor inhibition or prodrug thereof and (ii) an anticancer agent and/or an immunomodulatory agent; for use in treating and/or preventing leukemia.
Embodiment 23. Use of an inhibitor of R-spondin 2 and/or R-spondin 3 mediated membrane clearance of a BMP receptor in the manufacture of a pharmaceutical composition for treating and/or preventing leukemia.
Embodiment 24. The subject matter of any of the preceding embodiments, wherein said R-spondin is R-spondin 2.
Embodiment 25. The subject matter of any of the preceding embodiments, wherein said leukemia is acute myeloid leukemia (AML).
Embodiment 26. An inhibitor of R-spondin 2 and/or R-spondin 3 mediated bone morphogenetic protein (BMP) receptor inhibition for use in treating and/or preventing leukemia in a subject with an antiproliferative agent.
Embodiment 27. An antiproliferative agent for use in treating and/or preventing leukemia in a subject with an inhibitor of R-spondin 2 and/or R-spondin 3 mediated bone morphogenetic protein (BMP) receptor inhibition.
Embodiment 28. A combined preparation comprising an antiproliferative agent and an inhibitor of R-spondin 2 and/or R-spondin 3 mediated bone morphogenetic protein (BMP) receptor inhibition.
Embodiment 29. The combined preparation of embodiment 28 for use in medicine.
Embodiment 30. The combined preparation of embodiment 28 for use in treating and/or preventing leukemia in a subject.

All references cited in this specification are herewith incorporated by reference with respect to their entire disclosure content and the disclosure content specifically mentioned in this specification.

### FIGURE LEGENDS

FIG. 1: RSPO2 and -3 antagonize BMP4 signaling independently of WNT: a) BRE reporter assay in HEPG2 cells stimulated by BMP4, with or without RSPO1-4 treatment. b,c) Western blot analyses of phosphorylated-(pSmad1) and total Smad1 (tSmad1) in HEPG2 cells upon RSPO2 DNA transfection (b) or treatment with RSPO2 (c). d) qRT-PCR analysis of ID1 in HEPG2 cells upon BMP4, with or without overnight RSPO2 treatment. e) Domain structures of RSPO2 and deletion mutants. sp, signal peptide; FU, furin domain; TSP1, thrombospondin domain 1. f) BRE reporter assay in HEPG2 cells stimulated by BMP4+RSPO2 and deletion mutants.

FIG. 2: RSPO2 inhibits BMP4 signaling in Xenopus dorsoventral embryonic patterning: a) BMP-reporter assays with neurulae (St.15) injected as indicated. Data are biological replicates and displayed as means±SD with unpaired t-test. b) Quantification of in situ hybridization of BMP4 target sizzled in gastrula embryos (St.11) injected as indicated.

FIG. 3: RSPO2 reduces ALK3 surface levels via ZNRF3/RNF43: a) BRE reporter assay in HEPG2 cells treated with ALK3RD and RSPO1-4. b) In vitro binding assay between RSPO1-3, FGF and ALK3^{ECD}. c) Scatchard plot of RSPO2 and ALK3^{ECD} binding. d) Quantification of cell surface binding assay in HEK293T cells transfected with receptor DNAs, followed by RSPO binding as indicated. Data shows a representative from 4 independent experiments. e) Western blot analysis of ALK3 in stage 15 embryos injected as indicated. GFP, loading control. f) Western blot analysis in stage 18 embryos injected as indicated. g) BRE reporter assay in HEPG2 cells transfected with siRNA and treated with BMP4+RSPO2. h) BRE reporter assay in HEPG2 cells treated as indicated. i) In vitro binding assay for ZNRF3 and ALK3^{ECD} as indicated. j), Model for RSPO2-ZNRF3-ALK3 module (1) to mediate membrane clearance and degradation of ALK3 (2). Data for BRE reporter assays are biological replicates; binding assays are experimental replicates; ns, not significant, *P<0.05, **P<0.01, ***P<0.001, ****P<0.0001 from unpaired t-test. Western blots show representative results from three independent experiments.

FIG. 4: RSPO2 blocks macrophage differentiation by antagonizing BMP signaling.
a) qRT-PCR analysis of ID1 in THP-1 cells treated siRNA as indicated. b) Western blot analysis of phosphorylated Smad1 (pSmad1) and total Smad1 (tSmad1) in THP-1 cells treated with siRNA and BMP4 as indicated. c) Western blot analysis of pSmad1 and tSmad1 in siRNA treated THP-1 cells, with or without ALK inhibitor (LDN-193189, LDN) treatment. d) Western blot analyses of pSmad1 and tSmad1 in THP-1 cells treated with siRNA and BMP4 as indicated. e) Quantification of FACS analysis for CD14⁺- or CD11B⁺ cells upon siRNA transfection of THP-1 cells. f) qRT-PCR analysis of CD14 and CD11B expression in siRNA treated THP-1 cells. g) Quantification of FACS analysis for CD14⁺ cells in THP-1 cells following BMP4 stimulation. h-j) Quantification of FACS analysis for CD11⁺- or CD14B⁺ cells upon antibody treatment of THP-1 cells.

FIG. 5: Loss of RSPO2 in THP-1 cells increases BMP signaling and decreases cell growth: a) qRT-PCR analysis of indicated genes in THP-1 clones upon Dox treatment. b) Quantification of FACS analysis for CD11B⁺ cells in THP-1 clones treated as indicated. c) Cumulative cell growth curve depicted as the total cell count of THP-1 clones with Dox treatment. d) Representative images of CFC assay in THP-1 clones. e) Experimental scheme of THP-1 xenograft mice. f) Disease free survival (DFS) plot of THP-1 xenograft mice. g) qPCR analysis of the THP-1 burden in mice blood. h) Images show the livers and spleens harvested from THP-1 xenograft mice treated with or without Dox. Scale bar=1 cm. i) Quantification of FACS analysis for CD11B⁺ CD45⁺ cells in mice bone marrow. Data for FACS analyses are biological replicates and qRT-PCR are experimental replicates, and displayed as means±SD with unpaired t-test or one-way ANOVA test. Data show one representative of multiple independent experiments. ns, not significant; *P<0.05, **P<0.01, ***P<0.001, ****P<0.0001.

FIG. 6: High RSPO2 expression is a predictor for poor prognosis in AML: a-i) Kaplan-Meier plot of AML patients stratified by different gene expression levels (low and high according to the median). j) Table showing the number of patients analyzed (n), hazard ratio (HR), median survival ratio and significance of survival differences. ns, not significant; **p<0.01 from log-rank test. k-m) Kaplan-Meier plots of AML patients stratified by RSPO2 expression levels in the following cohorts: k) Beat AML (Tyner et al (2018), Nature 562 (7728): 526), 1) TARGET AML, and m) Leucegene AML.

FIG. 7: Loss of RSPO2 increases BMP signaling, induces differentiation and reduces CFU of MOLM14 AML cells; a) pSmad1 and tSmad1 levels shown by western blot with cell lysates from MOLM14 TetOn-shRNA clones; b) qRT-PCR analysis of RSPO2 and ID1 in MOLM14 TetOn-shRNA clones; c) Quantification of FACS analysis for CD11B+ cells in MOLM14 TetOn-shRNA clones; d) CFC assay with MOLM14 TetOn-shRNA clones.

FIG. 8: Loss of RSPO2 sensitize AML cells to chemotherapeutic drug cytarabine (AraC) treatment; a) and b) Cumulative cell growth depicted as the total cell count of THP-1 clones with Dox and AraC treatment.; c) AraC IC50 of THP-1 TetOn-shRNA clones upon Dox treatment.

The following Examples shall merely illustrate the invention. They shall not be construed, whatsoever, to limit the scope of the invention.

### EXAMPLE 1: MATERIALS AND METHODS

### 1.1 Constructs

Alkaline phosphatase (AP) fusions with RSPOs (human RSPO1^{ΔC}-AP-pCDNA3, RSPO2^{ΔC}-AP-pCDNA3, RSPO2^{ΔC}-AP-pCS2+, RSPO3^{ΔC}-AP-pCDNA3, murine RSPO4^{ΔC}-pCDNA3) were generated by replacing the C-terminal domain (ΔC) by AP and used to produce conditioned media. Human RSPO2 wild-type (RSPO2), Furin1 and Furin2 deletion mutants (RSPO2^{ΔFU}), and TSP1 domain deletion mutant (RSPO2^{ΔTSP}) are ORFs lacking the C-terminal domain, C-terminally tagged with a Flag-tag and subcloned into pCS2+. R1-TSP^{R2}, R1-TSP^{R2}-AP and R1-TSP^{R2}-Flag plasmids were generated by fusion PCR and cloned in pCS2+. Conditioned media from all RSPO constructs were adjusted to equal concentration by western blot and AP activity measurement, and further validated by WNT reporter assay using HEK293T cells. The extracellular domain of ALK3 (ALK3^{ECD}) was subcloned in AP-pCS2+ for generating conditioned medium and used in in vitro binding assays. Constitutively active forms of ALK2,3,6 (ALK^{QD}) were generated by Gln-Asp mutations as described in Fujii et al. (1999), Mol Biol Cell 10, 3801-3813, doi: 10.1091/mbc.10.11.3801. HA-tagged ALK3 was a gift from Dr. D. Koinuma (Goto et al. (2007), J Biol Chem 282, 20603-20611, doi: 10.1074/jbc.M702100200). For Xenopus mRNA microinjection, Xenopus laevis BMP4-pCS2+, myc-tagged RSPO2^{ΔC}-myc-pCS2+, RSPO2^{ΔFU}-myc-pCS2+ and RSPO2^{ΔTSP}-myc-pCS2+ plasmids, DNALK3-pCS2+, membrane-RFP, EYFP-tagged human ALK3-pCS2+ were used for in vitro transcription. Human ZNRF3 and ZNRF3^{ΔRING} constructs were gifts from Dr. F. Cong (Novartis; Hao et al. (2012), Nature 485, 195-200, doi: 10.1038/nature11019), and ORFs were subcloned in flag-pCS2+ for in vitro transcription.

A list of constructs used is provided in Table 1 below.

### 1.2 Cell Culture

HEK293T and HEPG2 cells (ATCC) were maintained in DMEM High glucose (Gibco 11960) supplemented with 10% FBS (Capricorn FBS-12A), 1% penicillin-streptomycin (Sigma P0781), and 2 mM L-glutamine (Sigma G7513). H1581 (gift from Dr. R. Thomas) and THP-1 cells (gift from Dr. S. Wiemann) were maintained in RPMI (Gibco 21875) with 10% FBS, 1% penicillin-streptomycin, 2 mM L-glutamine and 1 mM sodium pyruvate (Sigma S8636). Mycoplasma contamination was negative in all cell lines used. siRNAs and plasmids were transfected using DharmaFECT 1 transfection reagent (Dharmacon T-2001) and Lipofectamine 3000 (Invitrogen L3000) respectively, according to manufacturer protocols.

### 1.3 Generation of Conditioned Medium

HEK293T cells were seeded in 15 cm culture dishes and transiently transfected with RSPOs-AP, RSPOs-flag, ALK3^{ECD}-AP, DKK1 or WNT surrogate plasmids using X-tremeGENE9 DNA transfection reagent (Roche 06365809001). After 24 hours, media were changed with fresh DMEM, 10% FBS, 1% L-glutamine and 1% penicillin-streptomycin and cultured 6 days at 32° C. Conditioned media were harvested three times every two days, centrifuged and validated by TOPFlash assay or western blot analyses.

### 1.4 Luciferase Reporter Assays

BRE luciferase assays were executed using 300,000 ml⁻¹ of HEPG2 cells in 24-well plates. PGL3-BRE-Luciferase (500 ng ml⁻¹) and pRL-TK-Renilla plasmids (50 ng ml⁻¹) were transfected using Lipofectamine 3000. After 24 hours, cells were serum starved 2 hours and stimulated 14-16 hours with 80 ng ml⁻¹ recombinant human BMP4 protein (R&D systems 314-BP) along with AP tagged RSPO1-4 or flag tagged RSPO1-3 conditioned medium. Luciferase activity was measured with the Dual luciferase reporter assay system (Promega E1960). Firefly luminescence (BRE) was normalized to Renilla. TOPFlash luciferase assays were carried out as previously described (Berger et al. (2017), EMBO Rep 18, 712-725, doi: 10.15252/embr.201643585). Data are displayed as average of biological replicates with SD. Statistical analyses were made with the PRISM7 software using unpaired t-test or one-way ANOVA test. Not significant (ns) P>0.05, *P<0.05, **P<0.01, ***P<0.001, and ****P<0.0001.

### 1.5 Western Blot Analysis

Cultured cells were rinsed with cold PBS and lysed in Triton lysis buffer (20 mN Tris-Cl, pH 7.5, 1% Triton X-100, 150 mM NaCl, 1 mM EDTA, 1 mM EGTA, 1 mM β-glycerophosphate, 1 mM Na₃ VO₄) or RIPA buffer with complete Protease Inhibitor Cocktail (Roche 11697498001). Lysates were mixed with Laemmli buffer with β-mercaptoethanol and boiled at 95° C. for 5 min to prepare SDS-PAGE samples. Western blot images were acquired with SuperSignal West pico ECL (ThermoFisher 34580) or Clarity Western ECL (Biorad 1705061) using LAS-3000 system (FujiFilm). Quantification of blots was done using ImageJ software.

### 1.6 Cell Surface Biotinylation Assay

H1581 cells were seeded in 6 cm culture dishes and transfected with 50 nM of indicated siRNAs and 2 μg of ALK3-HA DNA. Surface proteins were biotinylated with 0.25 mg/ml sulfo-NHS-LC-LC-Biotin (ThermoFisher 21338) at 4° C. for 30 min. The reaction was quenched by 10 mM Monoethanolamine and cells were harvested and lysed with Triton X-100 lysis buffer. 200-300 μg of lysate was incubated with 20 μl streptavidin agarose (ThermoFisher 20359) to pull-down biotinylated surface proteins and subjected to Western blot.

1.7 Xenopus laevis and Xenopus tropicalis Experiments

All X. laevis and X. tropicalis experiments were approved by the state review board of Baden-Württemberg, Germany (permit number G-141-18) and executed according to federal and institutional guidelines and regulations. Developmental stages of the embryos were determined according to Nieuwkoop and Faber. No statistical analysis was done to adjust sample size before the experiments. No randomization of injection order was used during the experiments.

1.8 Xenopus laevis Whole-Mount In Situ Hybridization

Whole-mount in situ hybridizations of Xenopus embryos were performed using digoxigenin (DIG)-labeled probes. Antisense RNA probes against rspo2 and bmp4 were generated by in vitro transcription as previously described (Kazanskaya et al. (2004), Dev Cell 7, 525-534, doi: 10.1016/j.devcel.2004.07.019). Probes against alk3 and znrf3 were prepared using full-size of Xenopus alk3 ORF or znrf3 ORF as a template. Mo and mRNA injected embryos were collected at stage 11 (gastrula) or 32 (tadpole) for in situ hybridization. Images were obtained using AxioCam MRc 5 microscope (Zeiss). Embryos in each image were selected using Magnetic Lasso tool or Magic Wand tool of Adobe Photoshop CS6 software, and pasted into the uniform background color for presentation.

### 1.9 Xenopus Microinjection and Phenotype Analysis

In vitro fertilization, microinjection and culture of Xenopus embryos were performed as previously described (Gawantka et al. (1995), EMBO J 14, 6268-6279). X. laevis embryos were microinjected with reporter DNAs, in vitro transcribed mRNAs or antisense morpholino oligonucleotide (Mo) using Harvard Apparatus microinjection system. Mos for rspo2 (Kazanskaya et al (2004, loc. cit.), lrp6, chordin, bmp4, znrf3 and standard control were purchased from GeneTools. rspo2^{ΔTSP} Mo was designed based on rspo2 sequence. X. laevis 4-cell stage embryos were microinjected 5 nl per each blastomere equatorially and cultured until indicated stages. Equal amount of total mRNA or Mo were injected by adjustment with ppl or standard control Mo. Scoring of phenotypes was executed blind, and data are representative images from two independent experiments. Embryos in each image were selected using Magnetic Lasso tool or Magic Wand tool of Adobe Photoshop CS6 software, and pasted into the uniform background color for presentation. Statistical analyses show Chi-square tests.

1.10 Xenopus tropicalis CRISPR/Cas9-Mediated Mutagenesis

The 5′ region of genomic sequences from X. tropicalis chordin (NM_001142657.1) and noggin (NM_001171898.1) were searched for synthetic guide RNA (gRNA) targeting sites using an online prediction tool (crispr.cos.uni-heidelberg.de). Primers were designed for PCR-based gRNA template assembly. A primer lacking target sequences was used as control gRNA. PCR reactions were performed with Phusion Hot Start Flex DNA Polymerase (NEB M0535), followed by in vitro transcription using MEGAscript T7 Transcription Kit (Invitrogen AM1334). Embryos were microinjected at one to two-cell stages with a mixture of 50 μg of gRNA and 1 ng of recombinant Cas9 protein (Toolgen) per embryo. Injected embryos were cultured until stage 26, fixed with MEMFA and phenotypes were analyzed. Scoring of phenotypes was executed at stage 30 with blinding, and data are representative images from three independent experiments. Defects were categorized by the severity of ventralization. ‘Severe’ showed small head, enlarged ventral tissues and short body axis. ‘Mild’ showed one or two of the defects described above. ‘Normal’ showed no visible differences to the uninjected control. Statistical analyses show Chi-square test.

1.11 Xenopus tropicalis T7 Endonuclease I Assay

To validate CRISPR/Cas9-mediated genome editing, three embryos of each injection set were lysed at stage 30 for genotyping PCR reactions. All target sequences were amplified with Roti-Pol Hot-TaqS Mix (Roth 9248). After denaturation for 3 min at 94° C. and reannealing (ramp 0.1° C. per sec), the PCR products were incubated with 3 U of T7 Endonuclease I for 45 min at 37° C. Cleavage results were visualized on a 2% agarose gel.

1.12 Xenopus laevis Western Blot Analysis

Injected Xenopus embryos were harvested at stage 15 to 18, homogenized in NP-40 lysis buffer (2% NP-40, 20 mM Tris-HCl pH 7.5, 150 mM NaCl, 10 mM NaF, 10 mM Na3VO4, 10 mM sodium pyrophosphate, 5 mM EDTA, 1 mM EGTA, 1 mM PMSF, and complete Protease Inhibitor Cocktail with a volume of 20 μl per embryo. Lysates were cleared with CFC-113 (Honeywell 34874), followed by centrifugation (14,000 rpm, 10 min at 4° C.), boiling at 95° C. for 5 min with NuPAGE Sample Buffer. 0.5-1 embryos per lane were loaded for SDS-PAGE analysis.

### 1.13 In Vitro Binding Assay

High binding 96-well plates (Greiner M5811) were coated with 2 μg ml⁻¹ of recombinant human RSPO1 (Peprotech 120-38), RSPO2 (Peprotech 120-43), RSPO3 (Peprotech 120-44) or FGF8b (Peprotech 100-25) recombinant protein reconstituted in bicarbonate coating buffer (50 mM NaHCO₃, pH 9.6) overnight at 4° C. Coated wells were washed three times with TBST (TBS, 0.1% Tween-20) and blocked with 5% BSA in TBST for 1 hour at room temperature. 1.5 U ml⁻¹ of ALK3^{ECD}-AP or control conditioned medium was incubated overnight at 4° C. Wells were washed six times with TBST and bound AP activity was measured by the chemiluminescent SEAP Reporter Gene Assay kit (Abcam ab 133077). For ZNRF3-ALK3 binding assay, plates were coated with recombinant human ZNRF3 Fc Chimera protein (R&D systems 7994-RF). ALK3^{ECD}-AP was preincubated with RSPO2-Flag, RSPO2^{ΔTSP}-Flag conditioned medium or recombinant RSPO protein prior to treatment. Control conditioned medium and vesicles were used as control. Data show average chemiluminescent activities with SD from experimental triplicates. Statistical analyses show unpaired t-tests.

### 1.14 Immunofluorescence

150,000 H1581 cells were grown on coverslips in 12-well plates, followed by siRNA and DNA transfection. After 48 hours cells were fixed in 4% PFA for 10 min. Cells were treated with primary antibodies (1:250) overnight at 4° C., and secondary antibodies (1:500) and Hoechst dye (1:500) were applied for 2 hours at room temperature. Tyramide Signal Amplification for detecting RSPO-HRP was carried out as previously described^{13,23}. Quantification was executed using ImageJ. Dot plots show average and SD from every cells analyzed with unpaired t-test.

For X. laevis embryos, alk3-EYFP and membrane-RFP mRNAs were coinjected with the indicated mRNAs or Mos. Embryos were dissected for animal or ventrolateral explants at stage 9 or stage 11.5, respectively. Explants were immediately fixed with 4% PFA for 2 hours and mounted with Fluoromount-G (ThermoFisher 00495802). Images were obtained using LSM 700 (Zeiss). Data are representative images from two independent experiments. For quantification, Pearson's correlation coefficient for EYFP and RFP was analyzed using 16-30 random areas harboring 10 cells chosen from 6-10 embryos per each set. Dot plots show an average and SD from every plane analyzed with unpaired t-test.

### 1.15 Cell Surface Binding Assay

Cell surface binding assays were carried essentially out as previously described (Dosch et al. (1997), Development 124, 2325-2334). In brief, human ALK3-HA and Xenopus tropicalis LGR4 DNA were transfected in HEK293T cells, and incubated with 1.5 U ml⁻¹ conditioned media for 3 hours on ice. After several washes and crosslinking, cells were treated with 2 mM Levamisole for 20 min to inactivate endogenous AP activities and developed with BM-Purple (Sigma 11442074001). Cells were mounted with Fluoromount G. Images were obtained using LEICA DMIL microscope/Canon DS126311 camera.

### 1.16 Quantitative Real-Time PCR

Cultured cells were lysed in Macherey-Nagel RAI buffer containing 1% β-mercaptoethanol and total RNAs were isolated using NucleoSpin RNA isolation kit (Macherey-Nagel 740955). Reverse transcription and PCR amplification were performed. Primers used in this study are listed in Supplementary Table 1. Graphs show relative gene expressions to GAPDH. Data are displayed as mean with SD from multiple experimental replicates. Statistical analyses were performed using PRISM7 software with unpaired t-test or one-way ANOVA test.

1.17 FACS analysis

For analyzing macrophage differentiation of THP-1, cells were harvested, pelleted and resuspended in ice-cold blocking buffer (PBS supplemented with 1% BSA and 0.1% NaN₃). Cells were treated with Fc Receptor Binding Inhibitor as recommended by the manufacturer (eBioscience 14916173) and stained directly with FITC/APC-conjugated antibodies diluted in blocking buffer or with non-conjugated primary antibodies followed by fluorochrome-labeled secondary antibodies. Isotype-matched antibodies were used as controls. Dead cells were excluded by counterstaining with propidium iodide. For analyzing apoptosis of THP-1, cells were fixed in 4% PFA, permeabilized by MeOH and blocked with PBS supplemented with 1% BSA and 0.1% Tween-20. Cells were stained with anti-active Caspase-3 antibody and fluorochrome-labeled secondary antibody. FACS Samples were analyzed with FACSCalibur or FACSCanto (BD Biosciences). 10,000 events per samples were acquired, and results were processed with Cell Quest or FACSDiva software (BD Biosciences). For BMP4 stimulation, cells were treated with 5 and 25 ng ml⁻¹ recombinant human BMP4 protein (R&D systems 314-BP). 100, 300 and 1000 nM LDN 193185 (Tocris 6053) were used for rescue assay. For the THP-1 cell number quantification and differentiation validation in vivo, bone marrow cells were harvested from tibias of NSG mice and red blood cells were removed by ACK Lysing Buffer (Gibco A1049201). Cells were stained and analyzed as above. Here, 50,000 PI-negative events per samples were acquired.

### 1.18 RSPO2 Neutralization Assay

THP-1 cells were treated with 0.3, 1.0 and 3.0 μg ml⁻¹ goat polyclonal anti-RSPO2 antibodies (R&D systems AF3266) or goat polyclonal GFP antibodies (ABIN 100085). After 48 hours, medium was replaced including fresh antibodies—and incubated another 24 hours. Western blot analysis and FACS analyses were performed as discussed above.

1.19 Generation of Inducible shRNA Expressing Cell Line

The sequences of shRSPO2 and shControl (Table 1) were synthesized, inserted into the transfer plasmid Tet-pLKO-puro (Addgene 21915) and validated by sequencing. Lentivirus was produced with the 3^{rd} generation lentiviral system according to the protocol available at the Trono lab as described (www.epfl.ch/labs/tronolab/). THP-1 cells were infected with lentivirus with 8 μg ml⁻¹ Polybrene (Sigma TR-1003) and selected with 0.5 μg ml⁻¹ puromycin (Calbiochem 540411). Single cell clones were obtained by limiting dilutions. The shRNA expression of clones was validated by monitoring RSPO2 expression after doxycycline treatment (1.0 μg ml⁻¹) for 3 days.

### 1.20 Colony Formation Assay

1,000 cells per well of Dox-inducible THP-1 clones were seeded into 24-well plates in RPMI+10% FBS, 1% penicillin-streptomycin, 1 mM sodium pyruvate, 2 mM L-glutamine and 0.5% methylcellulose, with or without Doxycycline (1 μg ml⁻¹). After 8 days incubation, microscopic dark field images were taken using LEICA DMIL microscope/Canon DS126311 camera. Quantification was executed with ImageJ, and statistics shows unpaired t-test.

1.21 In Vivo Experiments with NSG Mouse

NOD SCID gamma (NSG) mice were recruited from the Center for Preclinical Research, DKFZ, Heidelberg. Mice were maintained at a 12 h light-dark cycle with unrestricted Kliba 3307 diet and water. Randomized mouse cohorts (n=6-7 mice/group) were treated with Dox or vehicle throughout the study. Treatment started 3 days prior to cell transplantation via drinking water consisting 1 mg/ml Dox and 5% saccharose, or 5% saccharose only. One day prior to transplantation, mice were sub-lethally irradiated on whole body (2 Gy of a 137Cs-source, Type OB. 58/9021; Buchler GmbH, Braunschweig) and treated with Baytril (25 mg/kg bodyweight, i.e. 25 mg/ml drinking water) for 2 weeks. 500,000 cells in 100 μl PBS were injected intravenously into the lateral tail vein of 7-8-week-old female mice. Besides regular health checks, mouse body weight was taken twice per week throughout the experiment. Heparinized blood was collected from the tail vein at days 9 and 22 after transplantation. Terminal blood collection was performed under isoflurane anesthesia followed by cervical dislocation. Necropsies were taken as indicated or when mice reached a stop criterion of the German Society of Laboratory Animal Sciences (GV-SOLAS), until here defined as survival. All mouse experiments were in accordance with the approved guidelines of the local Governmental Committee for Animal Experimentation (RP Karlsruhe, Germany, license G140/19).

1.22 qPCR Analysis for THP-1 Burden in NSG Mouse

Genomic DNA was isolated from mouse blood samples using NucleoSpin Tissue kit (Macherey-Nagel 740952). Quantitative PCR was done with human Alu element specific primers and corresponding Taqman probe (Funakoshi et al. (2017), Sci Rep 7, 13202, doi: 10.1038/s41598-017-13402-3). For each reaction, 25 ng genomic DNA was used. A standard curve was generated with genomic DNA extracted from NSG mice blood containing known numbers of THP-1 cells and used for converting qPCR fluorescent signals to actual cell numbers. Normal NSG mice blood and nuclease free water were used as negative controls.

### 1.23 Kaplan-Meier Plot of AML Patients

Kaplan-Meier plots of AML patients were generated using UCSC Xena database (xena.ucsc.edu) and the GDC TCGA Acute Myeloid Leukemia dataset (portal.gdc.cancer.gov/projects/TCGA-LAML). Expression levels of indicated genes were stratified into two or three groups according to the RNAseq data. Statistical analyses were done with the PRISM7 software using Log-rank test.

### EXAMPLE 2: RESULTS

### 2.1 RSPO2 and -3 Antagonize BMP4 Signaling Independently of WNT

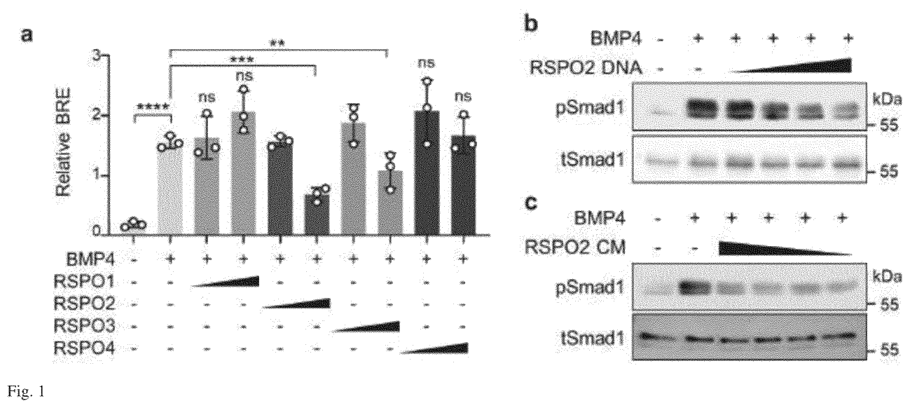

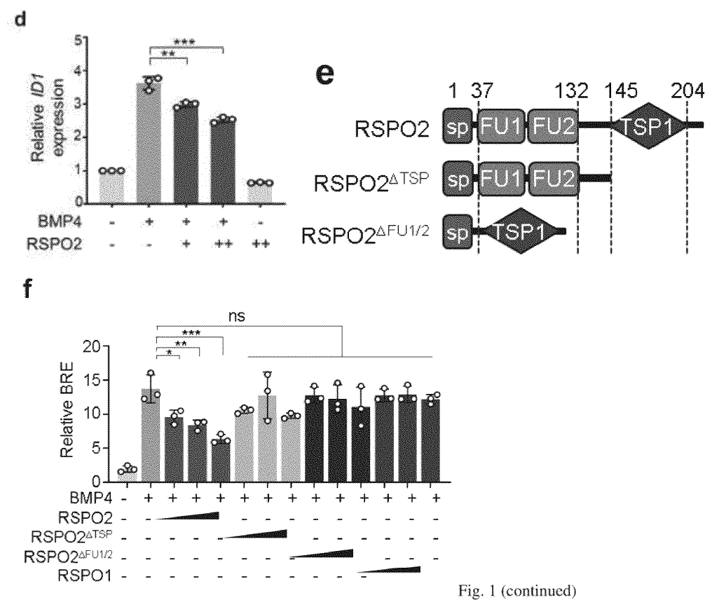

We tested if RSPO2 could suppress BMP signaling in human cells. To this end, we utilized human hepatocellular carcinoma (HEPG2) cells, which express very low levels of RSPOs. Intriguingly, treatment with RSPO2 and RSPO3 but not RSPO1 and RSPO4 decreased BMP4 signaling, while all RSPOs showed similar ability to amplify Wnt signaling (FIG. 1a). RSPO2 decreased phosphorylation of Smad1 and expression of ID1, which are hallmarks of BMP signaling activation (FIG. 1b-d). Importantly, inhibition of BMP signaling by RSPO2 was independent of both WNT/LRP6 and WNT/PCP signaling since it remained unaffected by siRNA knockdown of β-catenin, LGR4/5, LRP5/6, DVL1/2/3 and ROR1/2.

To delineate the domains required for BMP inhibition, we analyzed deletion mutants of RSPO2 and found both the TSP1- and FU-domains to be important (FIG. 1e-f). We next investigated RSPO2 deficiency in H1581 cells, a human large cell lung carcinoma cell line that expresses high levels of RSPO2. Knockdown of RSPO2 but not LRP5/6 sensitized H1581 cells to BMP stimulation.

### 2.2 RSPO2 Antagonizes BMP Signaling During Xenopus Embryonic Axis Development

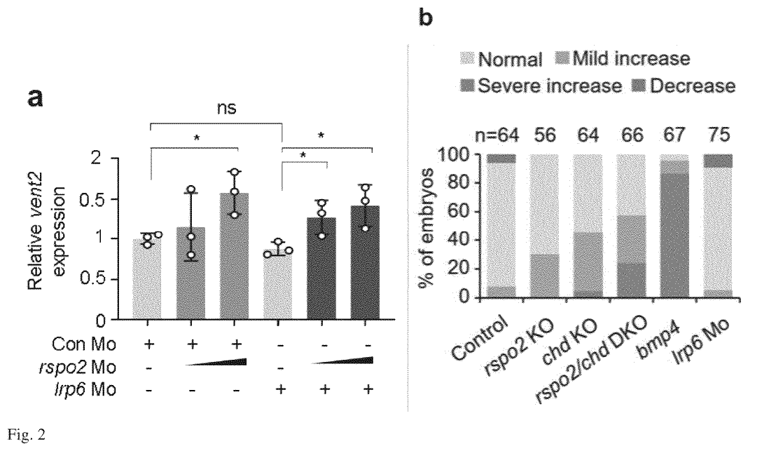

To analyze if RSPO2 inhibits BMP signaling in vivo, we turned our attention to Xenopus embryos, where BMP signaling plays a crucial role in dorsoventral patterning and rspo2 is required for WNT-mediated myogenesis (Kazanskaya et al. (2004, loc. cit.). Bmp4 overexpression ventralizes Xenopus embryos, resulting in small heads and enlarged ventral structures. Concordantly, injection of wild-type rspo2 mRNA, but not its deletion mutants, rescued these bmp4-induced malformations. Conversely, injection of a previously characterized rspo2 antisense Morpholino (Mo, Kazanskaya et al (2004), loc. cit.) increased endogenous BMP signaling (FIG. 2a). Strikingly, coinjection of bmp4 Mo and rspo2 Mo canceled each other out in BMP signaling reporter assays.

To confirm our morpholino data, we used a previously established guide RNA (gRNA, Szenker-Ravi et al. (2018), Nature 557, 564-569, doi: 10.1038/s41586-018-0118-y) to generate Crispr-Cas9-mediated Xenopus rspo2 knockout embryos. In accordance, rspo2 ablation increased BMP target gene expression (FIG. 2b) and yielded mildly ventralized embryos.

### 2.3 RSPO2 Bridges ALK3 and ZNRF3 and Triggers BMP Receptor Degradation

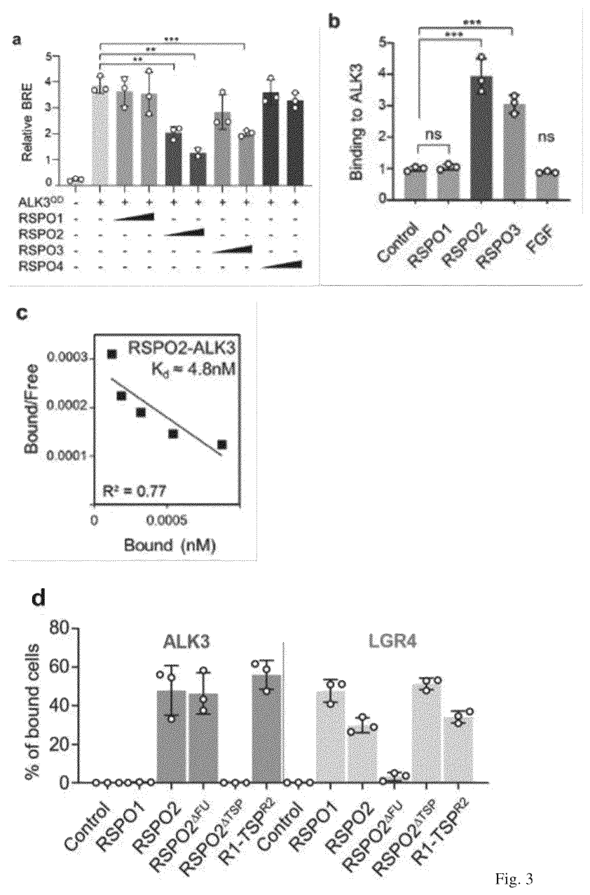

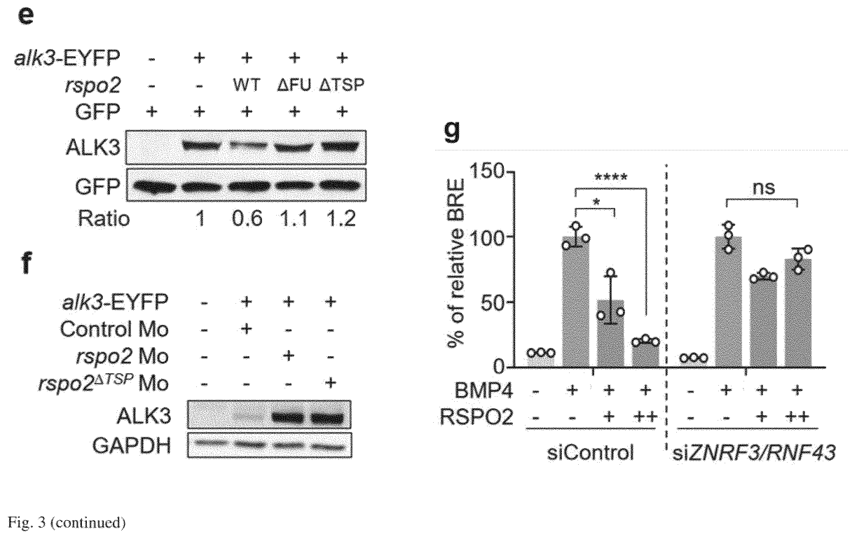

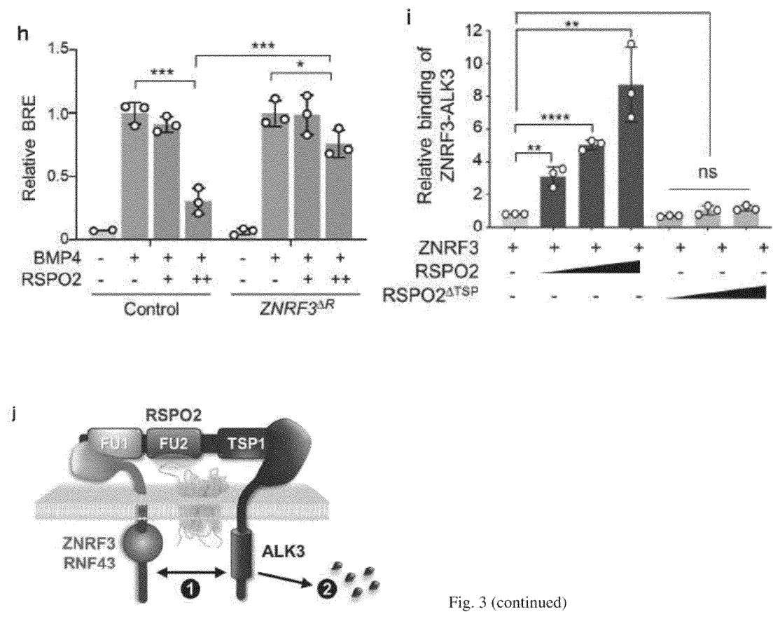

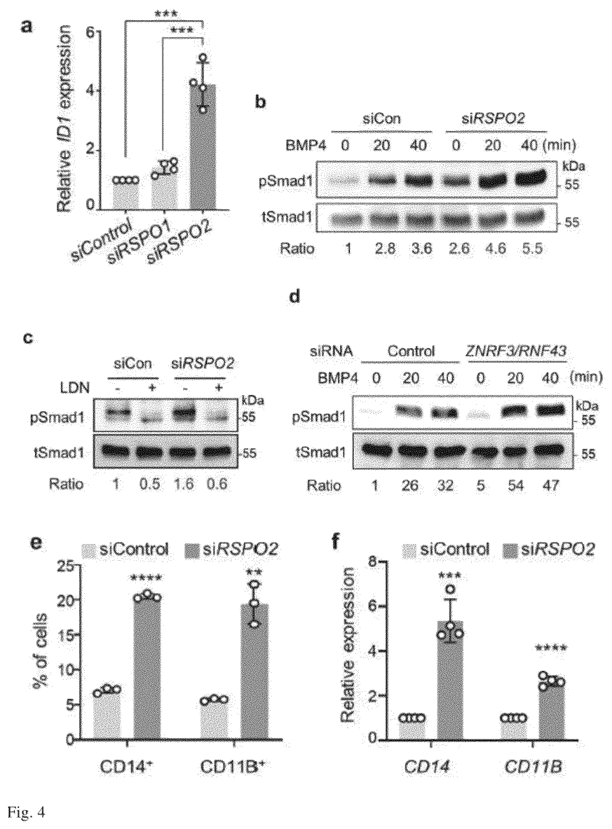

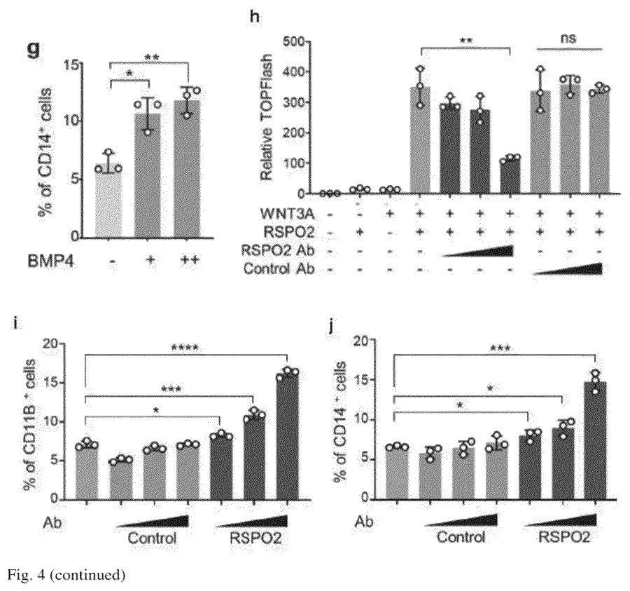

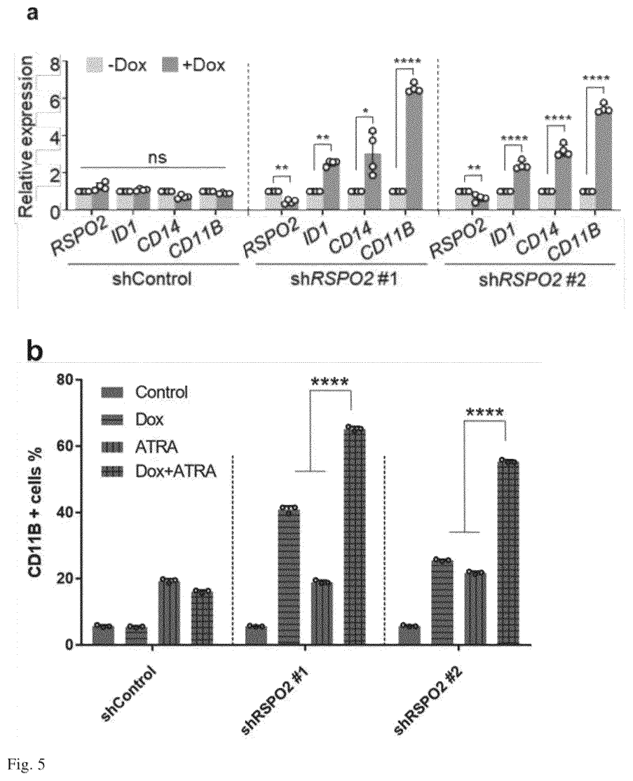

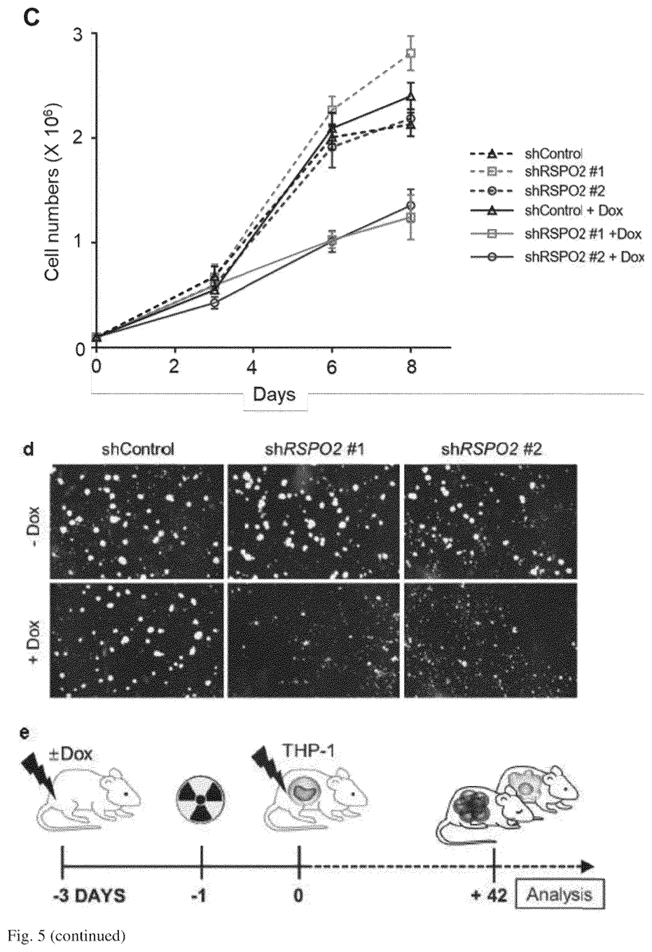

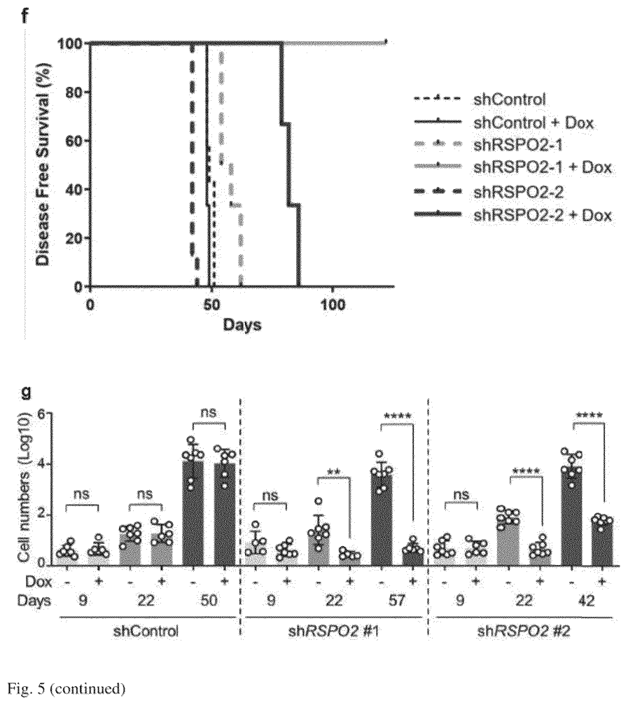

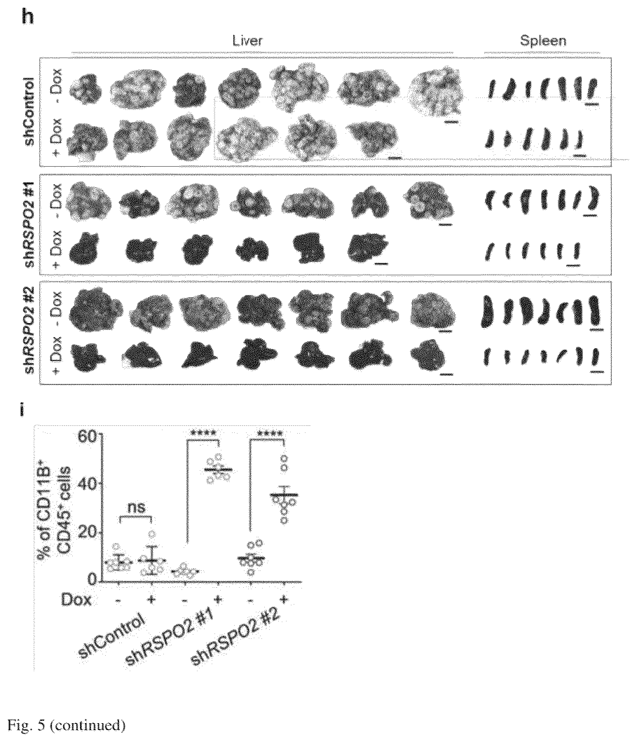

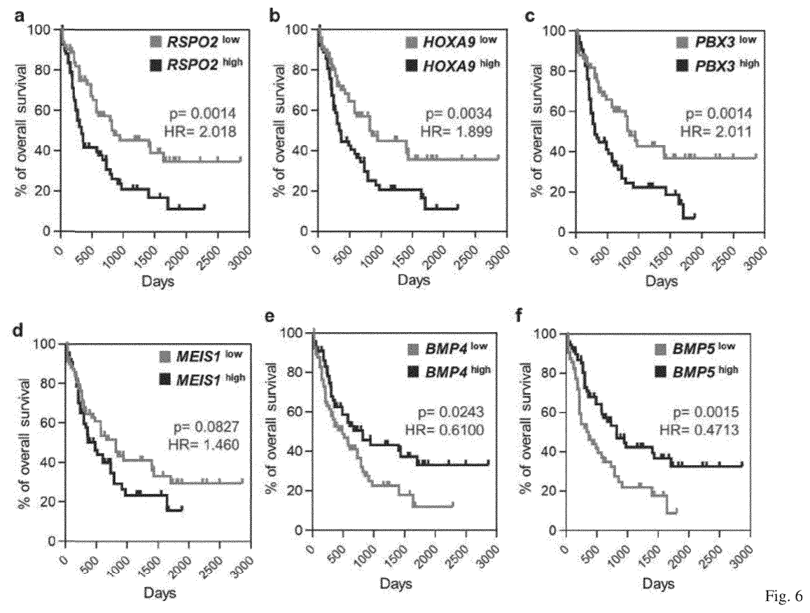

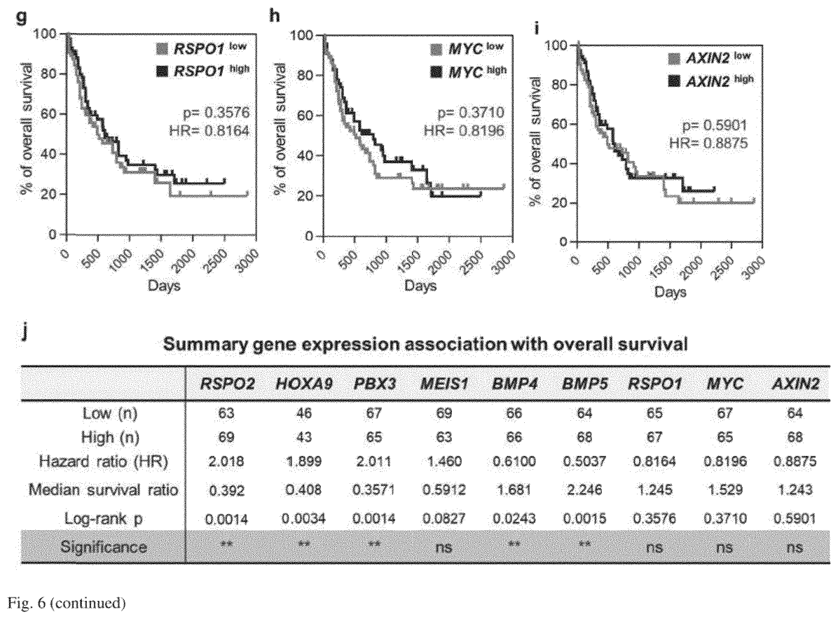

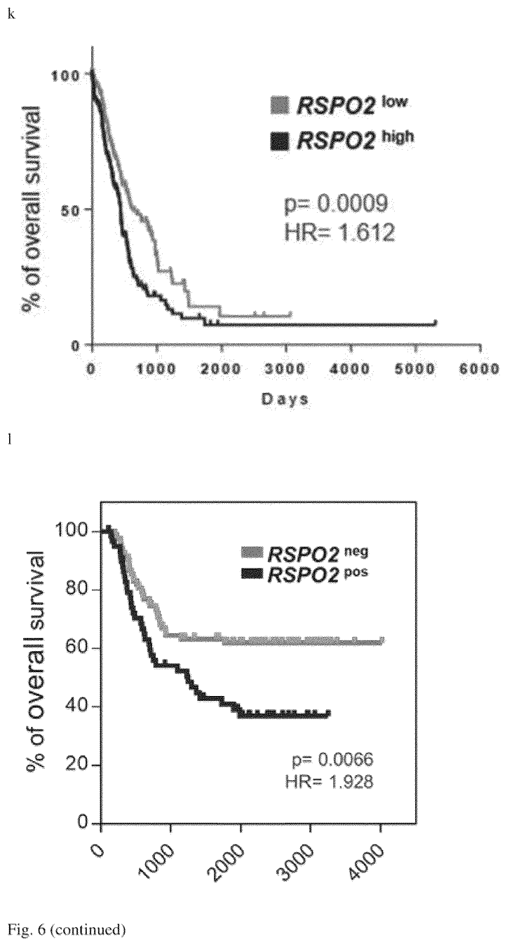

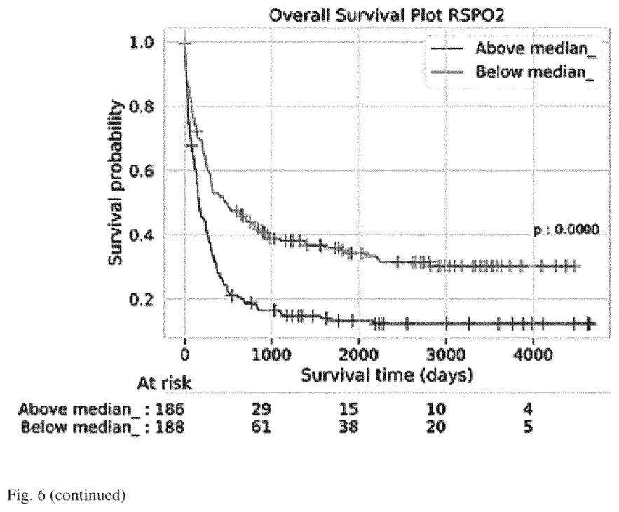

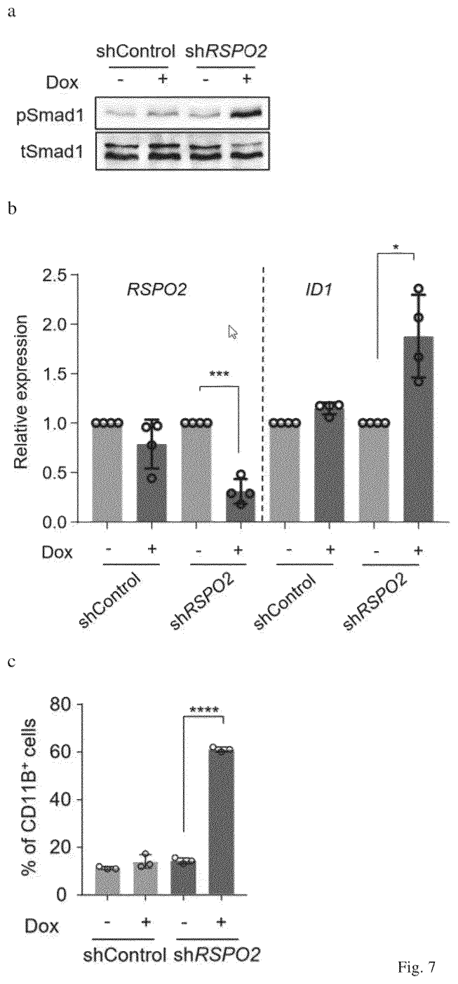

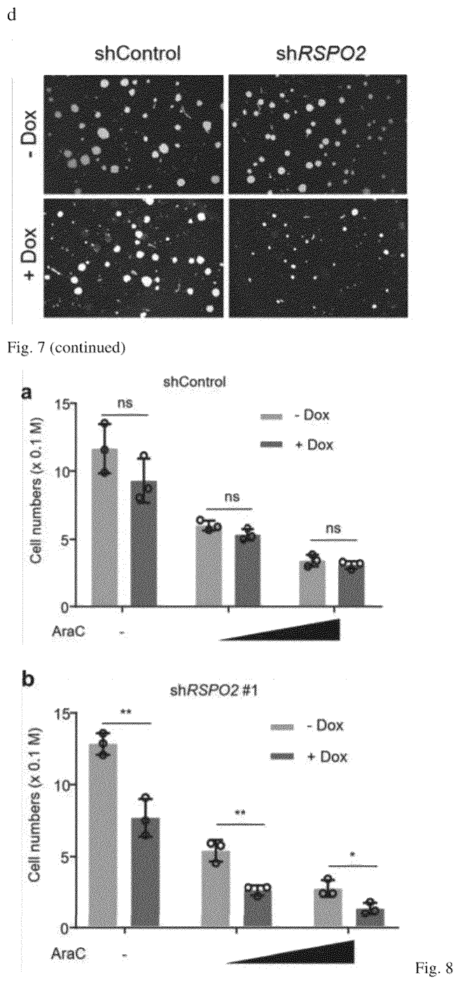

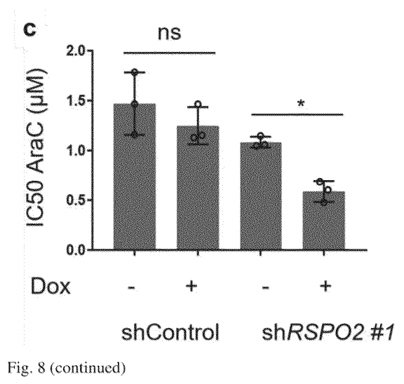

Given that RSPOs act by promoting receptor endocytosis, we postulated that RSPO2 might regulate BMP signaling through its receptors: ALK2, ALK3 and ALK6. To test this hypothesis, we analyzed the effects of RSPO2 on BMP signaling induced by constitutively active ALKs (ALK^{QD}). Interestingly, RSPO2 and -3 treatment specifically inhibited ALK3RD but not ALK2^{QD} or ALK6^{QD}. while RSPO1 and -4 had no effect (FIG. 3a). Indeed, in vitro binding assay revealed that RSPO2 bound the extracellular domain of ALK3 (FIG. 3b) with high affinity (K_{d}≈4.8 nM) (FIG. 3c). ALK3 binding required the TSP1—but not the FU domains of RSPO2, while, conversely, LGR binding required the FU domains but not TSP1 (FIG. 3d).

To investigate the consequence of this binding, we monitored ALK3 protein levels upon rspo2 overexpression or knockdown in Xenopus embryos, where alk3 is expressed from early stages onwards. rspo2 mRNA decreased protein levels from coinjected alk3-EYFP mRNA (FIG. 3e), while rspo2 MO increased ALK3-EYFP protein levels (FIG. 3f). Similarly, in H1581 cells siRSPO2 treatment increased ALK3 levels, altogether suggesting that RSPO2 destabilizes ALK3.

Our results indicate that the specificity for the RSPO-ALK3 interaction resides in the TSP1 domain of RSPOs. Consistently, the RSPO1 TSP1 domain shows only 43% and 50% sequence similarity to RSPO2 and RSPO3 respectively. We next asked whether TSP1-domain swapping could convey BMP inhibition to RSPO1. To this end, we generated an RSPO1 chimera (R1-TSP^{R2}) possessing the TSP1 domain of RSPO2. R1-TSP^{R2} activated WNT signaling and interacted with LGR4, similar to wt RSPO1. However, unlike wt RSPO1, R1-TSP^{R2} bound to ALK3 (FIG. 3d) and antagonized BMP signaling, mimicking the effects of RSPO2.

We next turned to the role of the FU domains in RSPO2, which are also required for inhibition of BMP signaling. FU1 and FU2 domains confer RSPO binding to ZNRF3/RNF43 and LGRs, respectively⁵. Knockdown of ZNRF3/RNF43 or expression of a dominant negative ZNRF3 (ZNRF3^{ΔR})¹⁵ prevented inhibition of BMP signaling by RSPO2 (FIG. 3g-h), supporting that RSPO2 requires ZNRF3/RNF43 to antagonize BMP signaling. Importantly, ZNRF3 interacted with ALK3 in the presence of RSPO2 but not RSPO2^{ΔTSP} or RSPO1 (FIG. 3i), indicating that RSPO2 bridges both transmembrane proteins. To test if RSPO2/ZNRF3 target ALK3 for endocytosis and lysosomal degradation, we treated cells with the clathrin inhibitor monodansylcadaverin (MDC), which eliminated inhibition of BMP signaling by RSPO2. Taken together, our results support a model (FIG. 3j) wherein RSPO2 bridges ZNRF3 and ALK3 and routes them towards clathrin-mediated endocytosis and lysosomal degradation, thereby antagonizing BMP signaling. We suggest that, based on our in vitro and in vivo data, a similar mechanism applies to RSPO3.

### 2.4 RSPO2 Maintains Cancer Cell Proliferation by Inhibiting BMP Signalling

To investigate the role of RSPO2 in macrophage maturation from precursors (monocytes), we utilized human monocytic leukemia THP-1 cells. THP-1 cells are used to model both monocyte to macrophage differentiation and AML (acute myeloid leukemia). In accordance with our observations in human cell lines and Xenopus, RSPO2—but not RSPO1 siRNA knockdown enhanced BMP signaling, as shown by induction of ID1 expression (FIG. 4a). Furthermore, RSPO2 knockdown was accompanied by increased Smad1-phosphorylation (FIG. 4b), and treatment with the BMP receptor inhibitor LDN 193189 reverted increased Smad1-phosphorylation (FIG. 4c). Consistently, siZNRF3/RNF43 enhanced Smad1-phosphorylation (FIG. 4d).

AML arises from uncontrolled proliferation and impaired differentiation of myeloid precursors (Nowak et al. (2009), Blood 113, 3655-3665, doi: 10.1182/blood-2009-01-198911), raising the possibility that RSPO2, via reducing BMP signaling and thereby inhibiting myeloid differentiation, could act as an endogenous oncogene in THP-1 cells. We therefore asked whether RSPO2 knockdown in THP-1 cells could induce monocyte-macrophage differentiation. Strikingly, siRSPO2 RNA treatment increased expression of the macrophage markers CD14 and CD11B (FIG. 4e-f), similar to BMP4 treatment (FIG. 4g). Attenuating BMP receptor signaling with LDN 193189 impaired monocyte-macrophage differentiation induced by siRSPO2. Neutralizing endogenous RSPO2 protein with an anti-RSPO2 antibody showed similar effects to siRSPO2 treatment (FIG. 4h-j). To test whether adding RSPO2 protein could impair monocyte-macrophage differentiation, we treated THP-1 cells with all-trans-retinoic acid (ATRA), which induces macrophage differentiation in a number of monocytic cell lines, including THP-1 cells. ATRA induced Smad1-phosphorylation as well as ID1 and CD11B expression, while co-treatment with RSPO2 reverted these effects. These results indicate that RSPO2 inhibits BMP signaling in THP-1 cells, thereby preventing macrophage differentiation and promoting self-renewal.

To further validate this conclusion, we established two THP-1 clones expressing Dox-inducible shRSPO2 RNA (TetOn-shRSPO2), where Dox-administration induced BMP signaling and robust monocyte-macrophage differentiation (FIG. 5 a). In addition, Dox+ATRA cooperated in inducing macrophage differentiation (FIG. 5 b). Consistent with RSPO2 maintaining a proliferative state, Dox treatment reduced THP-1 cell growth (FIG. 5c) and colony formation (FIG. 5d).

Also, loss of RSPO2 was found to increase BMP signaling, induce differentiation and reduce CFU of MOLM14 AML cells (FIG. 7).

Moreover, loss of RSPO2 was found to sensitize AML cells to chemotherapeutic drug cytarabine (AraC) treatment (FIG. 8).

To explore a putative therapeutic effect of inhibiting RSPO2 to restrict THP-1 growth in an AML xenograft model, we injected TetOn-shRSPO2 or -shControl THP-1 cells into immunodeficient (NSG) mice (FIG. 5e). Mice harboring TetOn-shControl THP-1 cells rapidly developed leukemia and succumbed within 50 days post-injection, with or without Dox treatment. Mice injected with the two TetOn-shRSPO2 THP-1 cell lines (#1, #2) also reached the humane endpoint between 45-62 days without Dox treatment. In contrast, upon Dox induction mice injected with cell line #2 survived almost twice as long and mice injected with line #1 where still alive at reporting time, corresponding to an at least 2.3-fold prolongation of disease-free survival (FIG. 5f). Extended survival was accompanied by reduced tumor burden (FIG. 5g-h) and enhanced macrophage differentiation in both cell lines (FIG. 5i).

Intriguingly, analysis of overall survival in AML patients in cancer databases revealed that high RSPO2 expression was a superior predictor for poor prognosis (hazard ratio 2.018; p=0.0014) compared to commonly used markers (HOXA9, hazard ratio 1.899; p=0.0034; PBX3, hazard ratio 2.011; p=0.0014; MEISI, hazard ratio 1.460; p=0.0304)^{31,32} (FIG. 6).

Our study reveals that RSPO2 and RSPO3 function not only to amplify WNT—but also to antagonize BMP signaling, and that ALK3 is a novel RSPO2/3 receptor. Given the importance of RSPOs as growth factors of normal and malignant stem cells this conclusion has important implications for disease beyond AML. For example, WNT activation accompanied by suppression of BMP signalling is a hallmark in colorectal cancer (CRC)³³. Hence, the dual function as WNT-activator and BMP suppressor sheds new light on how RSPO2 and RSPO3 gain-of-function mutations act as potent drivers in CRC (Seshagiri et al. (2012) Nature 488, 660-664, doi: 10.1038/nature11282). Our data also imply a function for ZNRF3 and possibly RNF43 in antagonizing BMP signalling, inviting a closer inspection of their loss-of-function phenotypes.

TABLE 1
Constructs used in the Examples
a. qRT-PCR primers

| Gene | Primer Sequence | SEQ ID NO |
|---|---|---|
| RSPO1 | F: ATCAAGGGGAAAAGGCAGA | 12 |
| | R: CAGAGCTCACAGCCTTTGG | 13 |
| RSPO2 | F: TGTCCAACCATTGCTGAATC | 14 |
| | R: TCCTCTTCTCCTTCGCCTTT | 15 |
| RSPO3 | F: AAGTGTCAGAAGGGAGAACGAG | 16 |
| | R: TGCTGTCAGGTATTGCTTCTTT | 17 |
| RSPO4 | F: TTTGGCCCACCAGAACAC | 18 |
| | R: CCGCAGGTCTTTCCATTG | 19 |
| ID1 | F: CCAGAACCGCAAGGTGAG | 20 |
| | R: GGTCCCTGATGTAGTCGATGA | 21 |
| ZNRF3 | F: TGTGCCATGTGTCTGGAGAA | 22 |
| | R: TTCCTGTGAAACCGGTGAGT | 23 |
| RNF43 | F: GTTTGCTGGTGTTGCTGAAA | 24 |
| | R: TGGCATTGCACAGGTACAG | 25 |
| GAPDH | F: AGCCACATCGCTCAGACAC | 26 |
| | R: GCCCAATACGACCAAATCC | 27 |
| CD14 | F: GTTCGGAAGACTTATCGACCAT | 28 |
| | R: ACAAGGTTCTGGCGTGGT | 29 |
| CD118 | F: AGGAAGCCGGAGAGGTCA | 30 |
| | R: CACAACACTCTGGATCTGTCCTT | 31 |

b. siRNAs

| siRNA | Dharmacon Catalogue # |
|---|---|
| siControl | D-001210-01-20 |
| sibcatenin | M-003482-00 |
| siLgr4 | M-003673-03 |
| siLgr5 | M-005577-01 |
| siLrp5 | M-003844-02 |
| siLrp6 | M-003845-03 |
| siRspo1 | M-018179-01 |
| siRspo2 | M-017888-01 |
| siZnrf3 | M-010747-02 |
| siRnf43 | M-007004-02 |

c. shRNAs

| Human | | |
|---|---|---|
| shRNA | Sequence | SEQ ID NO |
| shControl | ATCTCGCTTGGGCGAGAGTAAGctcgagCT | 32 |
| | TACTCTCGCCCAAGCGAGAT | |
| shRSPO2 | GACAATGGGTGTAGCCGATctcgagATCGG | 10 |
| | CTACACCCATTGTC | |

d. Morpholinos

| Gene | Sequences (5′-3′) | SEQ ID NO |
|---|---|---|
| rspo2 | GCCGTCCAAATGCAGTTTCAAC | 9 |
| chordin | 1: ACGTTCTGTCTCGTATAGTGAGCGT | 35 |
| | 2: ACAGCATTTTTGTGGTTGTCCCGAA | 36 |
| bmp4 | CAGCATTCGGTTACCAGGAATCATG | 37 |
| lrp6 | CCCCGGCTTCTCCGCTCCGACCCCT | 38 |
| znrf3 | AACATAATTTCCCAGTCCTCAGTGG | 39 |
| rspo2ΔTSP | CAGCCATCTGGGAAGGCAACAGAAA | 40 |

e. Primers for X.tropicalis sgRNAs

| Gene | Sequences (5′-3′) | SEQ ID NO |
|---|---|---|
| rspo2 | TGACTCCATAGTATCCAGGA | 11 |
| noggin | CCTGGGACTTAGAATAGACC | 33 |
| chordin | CTGCTGGTGTCTTAGATTGG | 44 |

f. Genotyping Primers for X.tropicalis

| Primer | Sequences (5′-3′) | SEQ ID NO |
|---|---|---|
| rspo2 (F) | GCAGTATAGCATTGAAGTGGGTC | 41 |
| rspo2 (R) | GACAGATTCGTTCATCACTACATAG | 42 |
| noggin (F) | GATCTCTGGCAAGAAATCGGGA | 43 |
| noggin (R) | GACAGGACAGAAGGTCTGG | 44 |
| chordin (F) | CTCAACTCTTTGGACTCACTGAG | 45 |
| chordin (R) | ATGTGGCATCCTAAGGATTTAGG | 46 |

### LITERATURE

Berger et al. (2017), EMBO Rep 18, 712-725, doi: 10.15252/embr.201643585
Dosch et al. (1997), Development 124, 2325-2334
Fujii et al. (1999), Mol Biol Cell 10, 3801-3813, doi: 10.1091/mbc.10.11.3801
Funakoshi et al. (2017), Sci Rep 7, 13202, doi: 10.1038/s41598-017-13402-3
Gawantka et al. (1995), EMBO J 14, 6268-6279
Goto et al. (2007), J Biol Chem 282, 20603-20611, doi: 10.1074/jbc.M702100200
Hao et al. (2012), Nature 485, 195-200, doi: 10.1038/nature11019
Hao et al. (2016), Cancers (Basel) 8, doi: 10.3390/cancers8060054
Kazanskaya et al. (2004), Dev Cell 7, 525-534, doi: 10.1016/j.devcel.2004.07.019
Nowak et al. (2009), Blood 113, 3655-3665, doi: 10.1182/blood-2009-01-198911
Sato et al. (2009), Nature 459, 262-265, doi: 10.1038/nature07935
Szenker-Ravi et al. (2018), Nature 557, 564-569, doi: 10.1038/s41586-018-0118-y
Tyner et al (2018), Nature 562 (7728): 526

1. A method for treating and/or preventing leukemia in a subject in need thereof, said method comprising administering an inhibitor of R-spondin 2 and/or R-spondin 3 mediated bone morphogenetic protein (BMP) receptor inhibition to said subject.

2. The method of claim 1, wherein said inhibitor is selected from the list consisting of an immunoglobulin or binding fragment thereof, a polypeptide comprising an isolated domain of said R-spondin, a polypeptide comprising an extracellular domain of a BMP receptor, an RNAi agent, a gRNA, a peptide aptamer, a polynucleotide aptamer, an anticalin, and a Designed Ankyrin Repeat Protein.

3. The method of claim 1, wherein said inhibitor is selected from
(i) an immunoglobulin or fragment thereof specifically binding to said R-spondin 2 or R-spondin 3, preferably to said R-spondin 2;
(ii) an immunoglobulin or fragment thereof specifically binding to the extracellular domain of a BMP receptor, preferably BMP receptor 1A;
(iii) a polypeptide comprising either the thrombospondin type-1 (TSP1) domain or the first furin-like (FU1) domain of said R-spondin, or a fragment thereof;
(iv) a polypeptide comprising an extracellular domain of a BMP receptor, preferably of BMP receptor 1A;
(v) any combination of (i) to (iv).

4. The method of claim 3, wherein said immunoglobulin or fragment thereof specifically binding to said R-spondin 2 or R-spondin 3 specifically binds the TSP1 domain and/or the FU1 domain of said R-spondin, preferably specifically binds the TSP1 domain of said R-spondin.

5. The method of claim 3, wherein said TSP1 domain corresponds to amino acids 147 to 204 of a human R-spondin 2, and/or wherein said FU1 domain corresponds to amino acids 37 to 84 of a human R-spondin 2.

6. The method of claim 3, wherein said immunoglobulin or subdomain thereof specifically binding to the extracellular domain of a BMP receptor specifically binds the activin receptor domain of said BMP receptor, preferably binds an epitope comprised in a peptide corresponding to amino acids 1 to 152 of the human BMP receptor 1A.

7. The method of claim 1, wherein said subject was identified as benefiting from treatment with an inhibitor of R-spondin 2 or R-spondin 3; and/or wherein said subject is suffering from a leukemia in which leukemia cells comprise a decreased activity of said BMP receptor, preferably caused by R-spondin 2 or R-spondin 3 overproduction.

8. The method of claim 7, wherein said inhibitor is retinoic acid, preferably all-trans retinoic acid.

9. A method for identifying a subject benefiting from leukemia treatment with an inhibitor of R-spondin 2 and/or R-spondin 3 mediated BMP receptor inhibition comprising
(a) contacting a sample comprising leukemia cells of said subject with an inhibitor of R-spondin 2 and/or R-spondin 3 mediated BMP receptor inhibition;
(b) determining the amount of BMP receptor, phospho-mothers against decapentaplegic homolog 1 (pSMAD1), DNA-binding protein inhibitor ID-1 (ID1), CD14, and/or integrin alpha-M (CD11B) in the leukemia cells of step (a),
(c) comparing the amount determined in step (b) to a reference, preferably from control treated leukemia cells of said subject, and
(d) based on the result of step (c), identifying a subject benefiting from treatment with an inhibitor of R-spondin 2 and/or R-spondin 3 mediated BMP receptor inhibition.

10. The method of claim 9, wherein said reference is derived from a population of apparently healthy subjects or from a population of subjects known not to benefit from treatment with an inhibitor of R-spondin 2 and/or R-spondin 3 mediated BMP receptor inhibition; and wherein a subject benefiting from treatment with an inhibitor of R-spondin 2 and/or R-spondin 3 mediated BMP receptor inhibition is identified if the amount determined in step (b) is higher than the reference; and/or wherein said reference is derived from a population of subjects known to benefit from treatment with an inhibitor of R-spondin 2 and/or R-spondin 3 mediated BMP receptor inhibition; and wherein a subject benefiting from treatment with an inhibitor of R-spondin 2 or R-spondin 3 mediated BMP receptor inhibition is identified if the amount determined in step (a) is equal to or higher than the reference.

11. (canceled)

12. (canceled)

13. A method for identifying a compound for treating and/or preventing leukemia, preferably acute myeloid leukemia (AML), comprising
(A) contacting leukemia cells, preferably AML cells, with a compound suspected to be a compound for treating leukemia,
(B) determining an amount of a BMP receptor, phospho-mothers against decapentaplegic homolog 1 (SMAD1), DNA-binding protein inhibitor ID-1 (ID1), CD14, integrin alpha-M (CD11B), R-spondin 2, and/or R-spondin 3 in said leukemia cells, and
(C) based on the result of step (B), identifying a compound for treating and/or preventing leukemia.

14. (canceled)

15. The method of claim 1, wherein said R-spondin is R-spondin 2 and/or wherein said leukemia is acute myeloid leukemia (AML).

16. The method of claim 1, further comprising administration of an antiproliferative agent to said subject.

17. (canceled)

18. A combined preparation comprising an antiproliferative agent and an inhibitor of R-spondin 2 and/or R-spondin 3 mediated bone morphogenetic protein (BMP) receptor inhibition.

19. (canceled)

20. (canceled)

21. The method of claim 1, comprising administration of a combined preparation comprising an antiproliferative agent and an inhibitor of R-spondin 2 and/or R-spondin 3 mediated bone morphogenetic protein (BMP) receptor inhibition to said subject.

22. The method of claim 9, wherein said R-spondin is R-spondin 2 and/or wherein said leukemia is acute myeloid leukemia (AML).


SEQUENCE LISTING (46 sequences)

SEQ ID NO: 1 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 2 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 3 - protein, 936 aa, Homo sapiens
MRPRSGGRPGATGRRRRRLRRRPRGLRCSRLPPPPPLPLLLGLLLAAAGPGAARAKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMGIFLAFFVVVSLVCLILLVKIKLKQRRSQNSMNRLAVQALEKMETRKFNSKSKGRREGSCGALDTLSSSSTSDCAICLEKYIDGEELRVIPCTHRFHRKCVDPWLLQHHTCPHCRHNIIEQKGNPSAVCVETSNLSRGRQQRVTLPVHYPGRVHRTNAIPAYPTRTSMDSHGNPVTLLTMDRHGEQSLYSPQTPAYIRSYPPLHLDHSLAAHRCGLEHRAYSPAHPFRRPKLSGRSFSKAACFSQYETMYQHYYFQGLSYPEQEGQSPPSLAPRGPARAFPPSGSGSLLFPTVVHVAPPSHLESGSTSSFSCYHGHRSVCSGYLADCPGSDSSSSSSSGQCHCSSSDSVVDCTEVSNQGVYGSCSTFRSSLSSDYDPFIYRSRSPCRASEAGGSGSSGRGPALCFEGSPPPEELPAVHSHGAGRGEPWPGPASPSGDQVSTCSLEMNYSSNSSLEHRGPNSSTSEVGLEASPGAAPDLRRTWKGGHELPSCACCCEPQPSPAGPSAGAAGSSTLFLGPHLYEGSGPAGGEPQSGSSQGLYGLHPDHLPRTDGVKYEGLPCCFYEEKQVARGGGGGSGCYTEDYSVSVQYTLTEEPPPGCYPGARDLSQRIPIIPEDVDCDLGLPSDCQGTHSLGSWGGTRGPDTPRPHRGLGATREEERALCCQARALLRPGCPPEEAGAVRANFPSALQDTQESSTTATEAAGPRSHSADSSSPGA

SEQ ID NO: 4 - protein, 532 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 5 - protein, 61 aa, Homo sapiens
GCEVGHWSEWGTCSRNNRTCGFKWGLETRTRQIVKKPVKDTILCPTIAESRRCKMTMRHCP

SEQ ID NO: 6 - protein, 48 aa, Homo sapiens
NPICKGCLSCSKDNGCSRCQQKLFFFLRREGMRQYGECLHSCPSGYYG

SEQ ID NO: 7 - protein, 152 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIR

SEQ ID NO: 8 - protein, 79 aa, Homo sapiens
KCYCSGHCPDDAINNTCITNGHCFAIIEEDDQGETTLASGCMKYEGSDFQCKDSPKAQLRRTIECCRTNLCNQYLQPTL

SEQ ID NO: 9 - DNA, 22 bp, Artificial Sequence
  R-spondin 2 morpholino
gccgtccaaatgcagtttcaac

SEQ ID NO: 10 - DNA, 44 bp, Artificial Sequence
  R-spondin 2 shRNA
gacaatgggtgtagccgatctcgagatcggctacacccattgtc

SEQ ID NO: 11 - DNA, 20 bp, Artificial Sequence
  R-spondin 2 gRNA
tgactccatagtatccagga

SEQ ID NO: 12 - DNA, 19 bp, Artificial Sequence
  RSPO1-F
atcaaggggaaaaggcaga

SEQ ID NO: 13 - DNA, 19 bp, Artificial Sequence
  RSPO1-R
cagagctcacagcctttgg

SEQ ID NO: 14 - DNA, 20 bp, Artificial Sequence
  RSPO2-F
tgtccaaccattgctgaatc

SEQ ID NO: 15 - DNA, 20 bp, Artificial Sequence
  RSPO2-R
tcctcttctccttcgccttt

SEQ ID NO: 16 - DNA, 22 bp, Artificial Sequence
  RSPO3-F
aagtgtcagaagggagaacgag

SEQ ID NO: 17 - DNA, 22 bp, Artificial Sequence
  RSPO3-R
tgctgtcaggtattgcttcttt

SEQ ID NO: 18 - DNA, 18 bp, Artificial Sequence
  RSPO4-F
tttggcccaccagaacac

SEQ ID NO: 19 - DNA, 18 bp, Artificial Sequence
  RSPO4-R
ccgcaggtctttccattg

SEQ ID NO: 20 - DNA, 18 bp, Artificial Sequence
  ID1-F
ccagaaccgcaaggtgag

SEQ ID NO: 21 - DNA, 21 bp, Artificial Sequence
  ID1-R
ggtccctgatgtagtcgatga

SEQ ID NO: 22 - DNA, 20 bp, Artificial Sequence
  ZNRF3-F
tgtgccatctgtctggagaa

SEQ ID NO: 23 - DNA, 20 bp, Artificial Sequence
  ZNRF3-R
ttcctgtgaaaccggtgagt

SEQ ID NO: 24 - DNA, 20 bp, Artificial Sequence
  RNF43-F
gtttgctggtgttgctgaaa

SEQ ID NO: 25 - DNA, 19 bp, Artificial Sequence
  RNF43-R
tggcattgcacaggtacag

SEQ ID NO: 26 - DNA, 19 bp, Artificial Sequence
  GAPDH-F
agccacatcgctcagacac

SEQ ID NO: 27 - DNA, 19 bp, Artificial Sequence
  GAPDH-R
gcccaatacgaccaaatcc

SEQ ID NO: 28 - DNA, 22 bp, Artificial Sequence
  CD14-F
gttcggaagacttatcgaccat

SEQ ID NO: 29 - DNA, 18 bp, Artificial Sequence
  CD14-R
acaaggttctggcgtggt

SEQ ID NO: 30 - DNA, 18 bp, Artificial Sequence
  CD11B-F
aggaagccggagaggtca

SEQ ID NO: 31 - DNA, 23 bp, Artificial Sequence
  CD11B-R
cacaacactctggatctgtcctt

SEQ ID NO: 32 - DNA, 50 bp, Artificial Sequence
  shControl
atctcgcttgggcgagagtaagctcgagcttactctcgcccaagcgagat

SEQ ID NO: 33 - DNA, 20 bp, Artificial Sequence
  noggin gRNA
cctgggacttagaatagacc

SEQ ID NO: 34 - DNA, 20 bp, Artificial Sequence
  Chordin gRNA
ctgctggtgtcttagattgg

SEQ ID NO: 35 - DNA, 25 bp, Artificial Sequence
  chordin morpholino 1
acgttctgtctcgtatagtgagcgt

SEQ ID NO: 36 - DNA, 25 bp, Artificial Sequence
  chordin morpholino 2
acagcatttttgtggttgtcccgaa

SEQ ID NO: 37 - DNA, 25 bp, Artificial Sequence
  bmp4 morpholino
cagcattcggttaccaggaatcatg

SEQ ID NO: 38 - DNA, 25 bp, Artificial Sequence
  lrp6 morpholino
ccccggcttctccgctccgacccct

SEQ ID NO: 39 - DNA, 25 bp, Artificial Sequence
  znrf3 morpholino
aacataatttcccagtcctcagtgg

SEQ ID NO: 40 - DNA, 25 bp, Artificial Sequence
  rspo2delTSP
cagccatctgggaaggcaacagaaa

SEQ ID NO: 41 - DNA, 23 bp, Artificial Sequence
  rspo2(F)
gcagtatagcattgaagtgggtc

SEQ ID NO: 42 - DNA, 25 bp, Artificial Sequence
  rspo2(R)
gacagattcgttcatcactacatag

SEQ ID NO: 43 - DNA, 22 bp, Artificial Sequence
  noggin(F)
gatctctggcaagaaatcggga

SEQ ID NO: 44 - DNA, 19 bp, Artificial Sequence
  noggin(R)
gacaggacagaaggtctgg

SEQ ID NO: 45 - DNA, 23 bp, Artificial Sequence
  chordin (F)
ctcaactctttggactcactgag

SEQ ID NO: 46 - DNA, 23 bp, Artificial Sequence
  chordin (R)
atgtggcatcctaaggatttagg